SEQUENCE LISTING (11 sequences)

SEQ ID NO: 1 - DNA, 1383 bp, nuc. & predicted a.a. seq. of mNR4
  CDS (61)..(1338)
  unsure (121): n=authors are unsure of exact sequence in this region
  unsure (122): n=authors are unsure of exact sequence in this region
  unsure (123): n=authors are unsure of exact sequence in this region
  unsure (640): n=authors are unsure of exact sequence in this region
  unsure (641): n=authors are unsure of exact sequence in this region
  unsure (642): n=authors are unsure of exact sequence in this region
tgaaaagatagaataaatggcctcgtgccgaattcggcacgagccgaggcgagggcctgcatggcgcggccagcgctgctgggcgagctgttggtgctgctactgtggaccgccaccgtgnnnggccaagttgccgcggccacagaagttcagccacctgtgacgaatttgagcgtctctgtcgaaaatctctgcacgataatatggacgtggagtcctcctgaaggagccagtccaaattgcactctcagatattttagtcactttgatgaccaacaggataagaaaattgctccagaaactcatcgtaaagaggaattacccctggatgagaaaatctgtctgcaggtgggctctcagtgtagtgccaatgaaagtgagaagcctagccctttggtgaaaaagtgcatctcaccccctgaaggtgatcctgagtccgctgtgactgagctcaagtgcatttggcataacctgagctatatgaagtgttcctggctccctggaaggaatacaagccctgacacacactatactctgtactattggtacagcagcctggagaaaagtcgtcaatgtgaaaacatctatagagaaggtcaacacattgcttgttcctttaaattgactaaagtggaacctnnnagttttgaacatcagaacgttcaaataatggtcaaggataatgctgggaaaattaggccatcctgcaaaatagtgtctttaacttcctatgtgaaacctgatcctccacatattaaacatcttctcctcaaaaatggtgccttattagtgcagtggaagaatccacaaaattttagaagcagatgcttaacttatgaagtggaggtcaataatactcaaaccgaccgacataatattttagaggttgaagaggacaaatgccagaattccgaatctgatagaaacatggagggtacaagttgtttccaactccctggtgttcttgccgacgctgtctacacagtcagagtaagagtcaaaacaaacaagttatgctttgatgacaacaaactgtggagtgattggagtgaagcacagagtataggtaaggagcaaaactccaccttctacaccaccatgttactcaccattccagtctttgtcgcagtggcagtcataatcctccttttttacctgaaaaggcttaagatcattatatttcctccaattcctgatcctggcaagatttttaaagaaatgtttggagaccagaatgatgataccctgcactggaagaagtatgacatctatgagaaacaatccaaagaagaaacggattctgtagtgctgatagaaaacctgaagaaagcagctccttgatggggagaagtgatttctttcttgccttcaatgtgaccctgt

SEQ ID NO: 2 - protein, 426 aa, nuc. & predicted a.a. seq. of mNR4
  unsure (21): authors are unsure about the sequence assignment
  unsure (194): authors are unsure about the sequence assignment
MARPALLGELLVLLLWTATVXGQVAAATEVQPPVTNLSVSVENLCTIIWTWSPPEGASPNCTLRYFSHFDDQQDKKIAPETHRKEELPLDEKICLQVGSQCSANESEKPSPLVKKCISPPEGDPESAVTELKCIWHNLSYMKCSWLPGRNTSPDTHYTLYYWYSSLEKSRQCENIYREGQHIACSFKLTKVEPXSFEHQNVQIMVKDNAGKIRPSCKIVSLTSYVKPDPPHIKHLLLKNGALLVQWKNPQNFRSRCLTYEVEVNNTQTDRHNILEVEEDKCQNSESDRNMEGTSCFQLPGVLADAVYTVRVRVKTNKLCFDDNKLWSDWSEAQSIGKEQNSTFYTTMLLTIPVFVAVAVIILLFYLKRLKIIIFPPIPDPGKIFKEMFGDQNDDTLHWKKYDIYEKQSKEETDSVVLIENLKKAAP

SEQ ID NO: 3 - DNA, 1383 bp, Human IL-13 receptor alpha-chain
  CDS (61)..(1338)
gagtctaacacggaccaaggagtttaacacgtgcggccgggttccgaggcgagaggctgcatggagtggccggcgcggctctgcgggctgtgggcgctgctgctctgcgccggcggcgggggcgggggcgggggcgcgcctacggaaactcagccacctgtgacaaatttgagtgtctctgttgaaaacctctgcacagtaatatggacatggaatccacccgagggagccagctcaaattgtagtctatggtattttagtcattttggcgacaaacaagataagaaaatagctccggaaactcgtcgttcaatagaagtacccctgaatgagaggatttgtctgcaagtggggtcccagtgtagcaccaatgagagtgagaagcctagcattttggttgaaaaatgcatctcacccccagaaggtgatcctgagtctgctgtgactgaacttcaatgcatttggcacaacctgagctacatgaagtgttcttggctccctggaaggaataccagtcccgacactaactatactctctactattggcacagaagcctggaaaaaattcatcaatgtgaaaacatctttagagaaggccaatactttggttgttcctttgatctgaccaaagtgaagcagtccagttttgaacaacacagtgtccaaataatggtcaaggataatgcaggaaaaattaaaccatccttcaatatagtgcctttaacttcccgtgtgaaacctgatcctccacatattaaaaacctctccttccacaatgatgacctatatgtgcaatgggagaatccacagaattttattagcagatgcctattttatgaagtagaagtcaataacagccaaactgagacacataatgttttctacgtccaagaggctaaatgtgagaatccagaatttgagagaaatgtggagaatacatcttgtttcatggtccctggtgttcttcctgatactttgaacacagtcagaataagagtcaaaacaaataagttatgctatgaggatgacaaactctggagtaattggagccaagaaatgactatagttaagaagcgcaattccacactctacataaccatgttactcattgttccagtcatcgtcgcaggtgcaatcatagtactcctgctttacctaaaaaggctcaagattattatattccctccaattcctgatcctggcaagatttttaaagaaatgtttggagaccagaatgatgatactctgcactggaagaagtacgacatctatgagaagcaaaccaaggaggaaaccgactctgtagtgctgatagaaaacctgaagaaagcctctcagtgatggagataatttatttttaccttcactgtgaccttgagaaga

SEQ ID NO: 4 - protein, 426 aa, Human IL-13 receptor alpha-chain
MEWPARLCGLWALLLCAGGGGGGGGAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKQSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMTIVKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 5 - protein, 30 aa, signal sequence of murine IL-3
MVLASSTTSIHTMLLLLLMLFHLGLQASIS

SEQ ID NO: 6 - protein, 8 aa, N-terminal FLAG epitope-tag
DYKDDDDK

SEQ ID NO: 7 - DNA, 31 bp, Oligo 1478 5′
agcttctagaacagaagttcagccacctgtg

SEQ ID NO: 8 - DNA, 30 bp, Oligo 1480 5′
aactccaccttctacaccacctgatctaga

SEQ ID NO: 9 - protein, 5 aa, NR4 Motif
  Unsure: Xaa may be any amino acid
WSXWS

SEQ ID NO: 10 - protein, 27 aa, N-term amino acid sequence of mNR4 (major)
  Unsure: Xaa may be any amino acid
DYKDDDDYKDDDESRTEVQPPVTXLSV

SEQ ID NO: 11 - protein, 27 aa, N-term amino acid sequence of mNR4 (minor)
  Unsure: Xaa may be any amino acid
ASISSSDYKDDDESRTEVQPPVTXLSV

(19) United States
(12) Patent Application Publication
(10) Pub. No.: US 2002/0090682 A1
(43) Pub. Date: Jul. 11, 2002

(54) Novel haemopoietin receptor and genetic sequences encoding same

(76) Inventors: Tracy Willson, North Balwyn (AU); Nicos A. Nicola, Mont Albert (AU); Douglas J. Hilton, Warrandyte (AU); Donald Metcalf, Balwyn (AU); Jian Guo Zhang, Hoppers Crossing (AU)

Correspondence Address: SCULLY, SCOTT, MURPHY & PRESSER, 400 Garden City Plaza, Garden City, NY, 11530, US

(21) Appl. No.: 10/036,568

(22) Filed: Nov. 7, 2001

Related U.S. Application Data
(63) Continuation of application No. 09/051,843, filed on Jun. 29, 1998.
Application No. 09/051,843 is a 371 of international application No. PCT/AU96/00668, filed on Oct. 23, 1996.

(30) Foreign Application Priority Data
Oct. 23, 1995 (AU) PN 6135
Dec. 22, 1995 (AU) PN 7276
Sep. 9, 1996 (AU) PO 2208

(51) Int. Cl. C07K 14/715; C12P 21/02; C12N 5/06; C07H 21/04

(52) U.S. Cl. 435/69.1; 435/325; 435/320.1; 530/350; 536/23.5

(57) ABSTRACT

The present invention relates generally to a novel haemopoietin receptor or components or parts thereof and to genetic sequences encoding same. The receptor molecules and their components and/or parts and the genetic sequences encoding same of the present invention are useful in the development of a wide range of agonists, antagonists, therapeutics and diagnostic reagents based on ligand interaction with its receptor.

The present invention relates generally to a novel haemopoietin receptor or components or parts thereof and to genetic sequences encoding same. The receptor molecules and their components and/or parts and the genetic sequences encoding same of the present invention are useful in the development of a wide range of agonists, antagonists, therapeutics and diagnostic reagents based on ligand interaction with its receptor.

Bibliographic details of the publications numerically referred to in this specification are collected at the end of the description. Sequence Identity Numbers (SEQ ID NOs.) for the nucleotide and amino acid sequences referred to in the specification are defined following the bibliography.

Throughout this specification and the claims which follow, unless the context requires otherwise, the word “comprise”, or variations such as “comprises” or “comprising”, will be understood to imply the inclusion of a stated integer or group of integers but not the exclusion of any other integer or group of integers.

The preferred haempoietin receptor of the present invention is referred to herein as “NR4”. The NR4 receptor interacts with IL-13 and is referred to herein as the IL-13 receptor or more particularly the IL-13 receptor α-chain (IL-13Rα). These terms are used interchangeably throughout the subject specification. The species from which a particular NR4 is derived is given in single letter abbreviation. For example, murine is “M” and human is “H”. A recombinant form may have the prefix “r”.

The rapidly increasing sophistication of recombinant DNA techniques is greatly facilitating research into the medical and allied health fields. Cytokine research is of particular importance, especially as these molecules regulate the proliferation, differentiation and function of a wide variety of cells. Administration of recombinant cytokines or regulating cytokine function and/or synthesis is increasingly becoming the focus of medical research into the treatment of a range of disease conditions.

Despite the discovery of a range of cytokines and other secreted regulators of cell function, comparatively few cytokines are directly used or targeted in therapeutic regimes. One reason for this is the pleiotropic nature of many cytokines. For example, interleukin (IL)-11 is a functionally pleiotropic molecule (1, 2), initially characterized by its ability to stimulate proliferation of the IL-6-dependent plasmacytoma cell line, T11 65 (3). Other biological actions of IL-11 include induction of multipotential haemopoietin progenitor cell proliferation (4, 5, 6), enhancement of megakaryocyte and platelet formation (7, 8, 9, 10), stimulation of acute phase protein synthesis (11) and inhibition of adipocyte lipoprotein lipase activity (12, 13).

Interleukin-13 (IL-13) is another important cytokine which shares a number of structural characteristics with interleukin-4 (IL4) [reviewed in 14 and 15]. The genes for IL-4 and IL-13 have a related intron/exon structure and are located close together on chromosome 5 in the human and the syntenic region of chromosome 11 in the mouse (14, 15). At the protein level, IL4 and IL-13 share approximately 30% amino acid identity, including four cysteine residues. Biologically, IL-13 and IL4 are also similar, being produced by activated T-cells and acting upon, for example, macrophages to induce differentiation and suppress the production of inflammatory cytokines. Additionally, human L-13 may act as a co-stimulatory signal for B-cell proliferation and affect immunoglobulin isotype switching (14, 15). The diverse and pleiotropic function of IL-13 and other haemopoietic cytokines makes this group important to study, especially at the level of interaction of the cytokine with its receptors. Manipulation and control of cytokine receptors and of cytokine-receptor interaction is potentially very important in many therapeutic situations, especially where the target cytokine is functionally pleiotropic and it is desired to block certain functions of a target cytokine but not all functions. Research into IL-13 and its receptor has been hampered due to the inability to clone genetic sequences encoding all or part of the IL-13 receptor. In accordance with the present invention, genetic sequences have now been cloned encoding the IL-13 receptor α-chain, a receptor subunit which is also shared with the IL-4 receptor. The availability of these genetic sequences permits the development of a range of therapeutic and diagnostic agents capable of modulating or monitoring IL-13 activity as well as the activity of cytokines related to IL-13 at the level of structure or function. In accordance with the present invention, an example of a cytokine related in structure and function to IL-13 is IL-4.

Accordingly, one aspect of the present invention is directed to a nucleic acid molecule comprising a sequence of nucleotides encoding or complementary to a sequence encoding an haemopoietin receptor from an animal or a derivative of said receptor.

More particularly, the present invention provides an isolated nucleic acid molecule comprising a sequence of nucleotides encoding or complementary to a sequence encoding an animal haempoietin receptor or a derivative thereof, said receptor capable of interaction with IL-13 or a derivative of IL-13.

In a related embodiment, the present invention provides an isolated nucleic acid molecule comprising a sequence of nucleotides encoding or complementary to a sequence encoding an animal haempoietin receptor or a derivative thereof, wherein said receptor:

(i) is capable of interaction with IL-13 or its derivatives; and

(ii) is capable of interaction with a complex between IL-4 and IL-4 receptor α-chain.

In accordance with these embodiments, a derivative of IL-13 includes agonists, antagonists, antibodies and mimetics.

The present invention is also directed to a nucleic acid molecule comprising a sequence of nucleotides encoding or complementary to a sequence encoding an animal IL-13 receptor α-chain or a derivative thereof.

In a related embodiment, the present invention contemplates a nucleic acid molecule comprising a sequence of nucleotides encoding or complementary to a sequence encoding a component of an animal IL-4 receptor or a derivative thereof.

Preferably, the animal is a mammal or a species of bird. Particularly, preferred animals include humans, laboratory test animals (e.g. primates, mice, rabbits, hamsters, guinea pigs), livestock animals (e.g. sheep, goats, horses, pigs, cows, donkeys), companion animals (e.g. dogs cats), captive wild animals (e.g. foxes, kangaroos, dingoes) and poultry birds (e.g. chickens, geese, ducks) and game birds (e.g. emus, ostriches). Although the present invention is exemplified with respect to mice and humans, the scope of the subject invention extends to all animals and birds.

The present invention is predicated in part on an ability to identify members of the haemopoietin receptor family on the basis of sequence similarity. Based on this approach, a genetic sequence was identified in accordance with the present invention which encodes a haemopoitin receptor. The expressed genetic sequence is referred to herein as “NR4”. In accordance with the present invention, NR4 has an apparent molecular mass when synthesised by transfected COS cells of from about 50,000 to about 70,000 daltons, and more preferably from about 55,000 to about 65,000 daltons. NR4 binds to IL-13 specifically and with low affinity and is considered, therefore, to be an IL-13 receptor α-chain. Accordingly, the terms “NR4” and “IL-13 receptor α-chain” (or “IL-13 Rα”) are used interchangeably throughout the subject specification. Furthermore, IL-13 binding to its receptor has been found to be competitively inhibited by IL-4 or a component thereof in cells which express the IL-4 receptor α-chain and this may provide a method for controlling IL-13-receptor interaction and will also provide a basis for the preparation and construction of mimetics.

Another aspect of the present invention provides a nucleic acid molecule comprising a sequence of nucleotides encoding IL-13 receptor α-chain having an amino acid sequence as set forth in SEQ ID NO:2 or having at least about 50% similarity to all or part thereof Preferably, the percentage similarity is at least about 60%, more preferably at least about 70%, even more preferably at least about 80-85% and still even more preferably at least about 90-95% or greater. The reference to all or part of a sequence is intended to include defining a hybrid molecule comprising parts of two receptors. It is not intended to encompass single amino acids.

A further embodiment of the present invention contemplates a nucleic acid molecule comprising a sequence of nucleotides encoding the IL-13 receptor α-chain and having a nucleotide sequence substantially as set forth in SEQ ID NO:1 or having at least about 50% similarity to all or part thereof. Preferably, the percentage similarity is at least about 60%, more preferably at least about 70%, even more preferably at least about 80-85% and still even more preferably at least about 90-95% or greater.

Still another aspect of the present invention provides a nucleic acid molecule comprising a sequence of nucleotides encoding IL-13 receptor α-chain having an amino acid sequence as set forth in SEQ ID NO:4 or having at least about 50% similarity to all or part thereof. Preferably, the percentage similarity is at least about 60%, more preferably at least about 70%, even more preferably at least about 80-85% and still even more preferably at least about 90-95% or greater.

Yet still a further embodiment of the present invention contemplates a nucleic acid molecule comprising a sequence of nucleotides encoding the IL-13 receptor α-chain and having a nucleotide sequence substantially as set forth in SEQ ID NO:3 or having at least about 50% similarity to all or part thereof. Preferably, the percentage similarity is at least about 60%, more preferably at least about 70%, even more preferably at least about 80-85% and still even more preferably at least about 90-95% or greater.

Accordingly, the present invention extends to the sequence of nucleotides set forth in SEQ ID NO:1 or 3 or the sequence of amino acids set forth in SEQ ID NO:2 or 4 or single or multiple nucleotide or amino acid substitutions, deletions and/or additions thereto.

The present invention further extends to nucleic acid molecules capable of hybridising under low stringency conditions to the nucleotide sequence set forth in SEQ ID NO:1 or 3 or a complementary form thereof.

The present invention extends to recombinant haempoietin receptors and in particular recombinant NR4 and recombinant hybrids containing NR4. Preferred recombinant polypeptides interact with IL-13 with low affinity and even more preferably with high affinity.

In a particularly preferred embodiment polypeptide has at least two of the following characteristics:

(i) comprises an amino acid sequence substantially as set forth in SEQ ID NO:2 or SEQ ID NO:4 or having at least about 50% similarity to all or part thereof;

(ii) is encoded by a nucleotide sequence substantially as set forth in SEQ ID NO:1 or SEQ ID NO:3 or having at least about 50% similarity to all or part thereof;

(iii) interacts with IL-13 or its derivatives with at least low affinity; and

(iv) has a molecular weight of from about 50,000 to about 70,000 daltons as determined by Western blot analysis when expressed in COS cells.

In a related embodiment, the polypeptide has at least three of the following characteristics:

(i) comprises an amino acid sequence substantially as set forth in SEQ ID NO:2 or SEQ ID NO:4 or having at least about 50% similarity to all or part thereof;

(ii) is encoded by a nucleotide sequence substantially as set forth in SEQ ID NO:1 or SEQ ID NO:3 or having at least about 50% similarity to all or part thereof;

(iii) interacts with IL-13 or its derivatives with at least low affinity;

(iv) has a molecular weight of from about 50,000 to about 70,000 daltons as determined by Western blot analysis when expressed in COS cells;

(v) comprises an amino acid sequence derived from IL4 receptor α-chain; and

(vi) is capable of interaction with IL-13 which is competitively inhibited by IL-4 in cells which express an IL-4 receptor α-chain.

Reference herein to “recombinant haempoietin receptor”, “NR4”, “IL-13 receptor” or “IL-13” receptor α-chain”includes reference to derivatives thereof such as parts, fragments, portions, homologues, hybrids or analogues thereof. The derivatives may be functional or not or may be non-functional but immunologically interactive with antibodies to all or part of the receptor. Derivatives of the receptor also cover agonists or antagonists of receptor-ligand interaction. Function is conveniently defined by an ability of NR4 to interact with IL-13 or its derivatives or for soluble NR4 to compete with IL-13-induced activities of certain cells.

Particularly preferred derivatives contemplated by the present invention include derivatives of IL-13 receptor α-chain which are capable of binding IL-13 with high affinity or with IL-13 and IL4 with high affinity; derivatives also encompass chimeric molecules such as between IL-13 receptor α-chain and, for example, IL-4 receptor α-chain which also bind IL-13 with high affinity.

Other fusion or chimeric molecules contemplated by the present invention include those between NR4 and members of the haemopietin receptor family, receptor tyrosine kinases, TNF/NGF receptors and G protein-coupled receptors. For example, chimeras may be between NR4 and IL-13 binding protein, IL-4 receptor α-chain, IL-2 receptor γ-chain or receptors for other cytokines involved or implicated in asthma and allergy such as IL-5. Other important chimeras include NR4 and immunoglobulins or other molecules which allow targeting of NR4 to particular cells or tissues, NR4 and toxins and NR4 and growth factors.

Reference herein to a low stringency at 42° C. includes and encompasses from at least about 1% v/v to at least about 15% v/v formamide and from at least about 1M to at least about 2M salt for hybridisation, and at least about IM to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.1 5M salt for washing conditions.

Yet another aspect of the present invention provides a nucleic acid molecule comprising a sequence of nucleotides which encodes or is complementary to a sequence which encodes an IL-13 receptor α-chain, said nucleic acid molecule having a nucleotide sequence substantially as set forth in SEQ ID NO:1 or 3 or a nucleic acid molecule which encodes a structurally similar IL-13 receptor α-chain or a derivative thereof and which is capable of hybridising to the nucleotide sequence substantially as set forth in SEQ ID NO:1 or 3 or a complementary form thereof under low stringency conditions.

Still yet another aspect of the present invention is directed to a nucleic acid molecule comprising a sequence of nucleotides which encodes or is complementary to a sequence which encodes the IL-13 receptor α-chain having an amino acid sequence substantially as set forth in SEQ ID NO:2 or 4 or comprises a nucleotide sequence coding for an amino acid sequence having at least about 50% similarity to the sequence set forth in SEQ ID NO:2 or 4 and is capable of hybridising to the sequence set forth in SEQ ID NO:1 or 3 under low stringency conditions.

The nucleic acid molecules contemplated by the present invention are generally in isolated form and may be single or double stranded, linear or closed circle DNA (e.g. genomic DNA), cDNA or mRNA or combinations thereof such as in the form of DNA:RNA hybrids. In a particularly preferred embodiment, the nucleic acid molecules are in vectors and most preferably expression vectors to enable expression in a suitable host cell. Particularly useful host cells include prokaryotic cells, mammalian cells, yeast cells and insect cells. The cells may also be in the form of a cell line.

According to this aspect of the present invention there is provided an expression vector comprising a nucleic acid molecule encoding the IL-13 receptor α-chain as hereinbefore described, said expression vector capable of expression in a particular host cell.

Another aspect of the present invention contemplates a recombinant polypeptide comprising a sequence of amino acids substantially as set forth in SEQ ID NO:2 or 4 or having at least about 50% similarity to all or part thereof. Preferably, the percentage similarity is at least about 60%, more preferably at least about 70%, even more preferably at least about 80-85% and still even more preferably at least about 90-95% or greater.

The recombinant polypeptide contemplated by the present invention includes, therefore, components, parts, fragments, derivatives, homologues or analogues of the IL-13 receptor α-chain and is preferably encoded by a nucleotide sequence substantially set forth in SEQ ID NO:1 or 3 or a molecule having at least about 50% similarity to all or part thereof or a molecule capable of hybridising to the nucleotide sequence set forth in SEQ ID NO:1 or 3 or a complementary form thereof. The recombinant molecule may be glycosylated or non-glycosylated. When in glycosylated form, the glycosylation may be substantially the same as naturally occurring IL-13 receptor α-chain or may be a modified form of glycosylation. Altered or differential glycosylation states may or may not affect binding activity of the IL-13 receptor α-chain.

The recombinant IL-13 receptor α-chain may be in soluble form or may be expressed on a cell surface or conjugated or fused to a solid support or another molecule.

The present invention further contemplates a method for producing a recombinant polypeptide having at least two of the following characteristics:

(i) comprises an amino acid sequence substantially as set forth in SEQ ID NO:2 or SEQ ID NO:4 or having at least about 50% similarity thereto;

(ii) is encoded by a nucleotide sequence substantially as set forth in SEQ ID NO:1 or SEQ ID NO:3 or having at least about 50% similarity thereto;

(iii) interacts with IL-13 or its derivatives with at least low affinity and

(iv) has a molecular weight of from about 50,000 to about 70,000 daltons as determined by Western blot analysis when expressed in COS cells, said method comprising culturing cells comprising the genetic constructs of the present invention for a time and under conditions sufficient to express the nucleic acid molecule in said genetic construct to produce a recombinant polypeptide and isolating said recombinant peptide.

Another embodiment provides a method of producing a recombinant polypeptide having at least three of the following characteristics:

(i) comprises an amino acid sequence substantially as set forth in SEQ ID NO:2 or SEQ ID NO:4 or having at least about 50% similarity to all or part thereof;

(ii) is encoded by a nucleotide sequence substantially as set forth in SEQ ID NO:1 or SEQ ID NO:3 or having at least about 50% similarity to all or part thereof;

(iii) interacts with IL-13 or its derivatives with at least low affinity;

(iv) has a molecular weight of from about 50,000 to about 70,000 daltons as determined by Western blot analysis when expressed in COS cells;

(v) comprises an amino acid sequence derived from IL-4 receptor α-chain; and

(vi) is capable of interaction with IL-13 which is competitively inhibited by IL-4 in cells which express an IL-4 receptor α-chain.

said method comprising culturing cells comprising the fusion genetic constructs according to the present invention for a time and under conditions sufficient to express the nucleic acid molecule in said fusion genetic constructs to produce a recombinant polypeptide and isolating said recombinant polypeptide.

The present invention further extends to cells such as animal cells which express the above-mentioned recombinant polypeptides.

Another embodiment of the present invention provides chemical analogues of the recombinant IL-13 receptor α-chain.

As stated above, the present invention further contemplates a range of derivatives of NR4. Derivatives include fragments, parts, portions, mutants, hybrids (including fusion and chimeric molecules), homologues and analogues of the NR4 polypeptide and corresponding genetic sequence. In one preferred embodiment the derivatives bind IL-13 with high affinity. Other preferred derivatives act as agonists, antagonist or mimetics. Derivatives also include single or multiple amino acid substitutions, deletions and/or additions to NR4 or single or multiple nucleotide substitutions, deletions and/or additions to the genetic sequence encoding NR4. “Additions” to amino acid sequences or nucleotide sequences include fusions with other peptides, polypeptides or proteins or fusions to nucleotide sequences. Reference herein to “NR4” includes reference to all derivatives thereof including functional derivatives or “NR4” immunologically interactive derivatives. The present invention also extends to hybrid molecules, such as between murine or human NR4 or derivatives thereof. A particularly preferred hybrid comprises NR4 and IL-4 receptor α-chain.

Analogues of NR4 contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzote, 4-chloromercuriphenylsulphonic acid, phenylmercury chloride, 2-chloromercuri4-nitrophenol and other mercurials; carbamoylation with cyanate at alkaline pH.

Tryptophan residues may be modified by, for example, oxidation with N-bromosucciinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 1.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH).

In addition, peptides can be conformationally constrained by, for example, incorporation of C_{α} and N_{α} -methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

These types of modifications may be important to stabilise NR4 if administered to an individual or for use as a diagnostic reagea

The present invention further contemplates chemical analogues of NR4 capable of acting as antagonists or agonists of NR4 or which can act as functional analogues of NR4. Chemical analogues may not necessarily be derived from NR4 but may share certain conformational similarities. Alternatively, chemical analogues may be specifically designed to mimic certain physiochemical properties of NR4. Chemical analogues may be chemically synthesised or may be detected following, for example, natural product screening.

The identification of NR4 permits the generation of a range of therapeutic molecules capable of modulating expression of NR4 or modulating the activity of NR4. Modulators contemplated by the present invention includes agonists and antagonists of NR4 gene expression or NR4 protein activity. Antagonists of NR4 gene expression include antisense molecules, ribozymes and co-suppression molecules. Agonists include molecules which increase promoter ability or interfere with negative regulatory mechanisms. Agonists of NR4 protein include antibodies, ligands and mimetics. Antagonists of NR4 include antibodies and inhibitor peptide fragments. Where a cell co-expresses NR4 and IL-4 receptor α-chain, agonists and antagonists may target the IL-4 receptor α-chain.

TABLE 1

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methythistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(ρ-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Other derivatives contemplated by the present invention include a range of glycosylation variants from a completely unglycosylated molecule to a modified glycosylated molecule. Altered glycosylation patterns may result from expression of recombinant molecules in different host cells.

Another embodiment of the present invention contemplates a method for modulating expression of the NR4 gene in a human, said method comprising contacting the NR4 gene encoding NR4 with an effective amount of a modulator of NR4 expression for a time and under conditions sufficient to up-regulate or down-regulate or otherwise modulate expression of NR4. A nucleic acid molecule encoding NR4 or a derivative thereof may also be introduced into a cell to enhance or alter NR4 related activities of that cell including replacing an endogenous NR4 gene sequence which may, for example, be defective or carry one or more undesired mutations. Conversely, NR4 antisense sequences (or sense sequences for co-suppression) such as oligonucleotides may be introduced to decrease NR4-related activies of any cell expressing the endogenous NR4 gene. Ribozymes may also be used.

Another aspect of the present invention contemplates a method of modulating activity of NR4 in a human, said method comprising administering to said mammal a modulating effective amount of a molecule for a time and under conditions sufficient to increase or decrease NR4 activity. The molecule may be a proteinaceous molecule or a chemical entity and may also be a derivative of NR4 or its ligand or a chemical analogue or truncation mutant of NR4 or its ligand. For example, IL-13 and IL-4 have been impliciated in the modulation of immune responses and in the production of IgE which is the immunoglobulin isotype associated with allergic or atopic diseases such as asthma. Modulating interactions between IL-13/IL-4 and their receptors may be important in treating inflammatory conditions such as allergic conditions. Elevated levels of IL-4/IL-13 and IgE are also important in diseases such as nephrotic syndrome, vernal and keratoconjunctivitis. Other diseases, the treatment of which is contemplated herein include bronchial asthma, perennial rhinitis and atopic dermatitis. Other disease conditions for which modulation of IL-13-receptor interaction may be important includes those conditions where IL-13 induces cytokine formation which in turn are involved in onset, progression and/or severity of diseases. Similarly, modulating IL-4-receptor interaction may also be important in controlling disease conditions. For example, some cancers may be exacerbated by the cytokine IL-13 or IL-4 which induce repressive immune effects or effector molecules which in turn reduce the body's ability to respond to the growth of the cancers.

Accordingly, the present invention contemplates a pharmaceutical composition comprising NR4 or a derivative thereof or a modulator of NR4 expression or NR4 activity and one or more pharmaceutically acceptable carriers and/or diluents. These components are referred to as the “active ingredients”.

In this regard there is provided a pharmaceutical composition comprising a recombinant haemopoietin receptor as hereinbefore described or a ligand (e.g. IL-13) binding portion thereof and one or more pharmaceutically acceptable carriers and/or diluents.

In another embodiment, there is provided a pharmaceutical composition comprising a ligand (e.g. IL-13) to the recombinant haemopoietin receptor as hereinbefore described and one or more pharmaceutically acceptable carriers and/or diluents. Still a further aspect of the present invention contemplates a method of treatment of an animal comprising administering to said animal a treatment effective amount of a recombinant haemopoietin receptor as hereinbefore described or a ligand binding portion thereof or a ligand (e.g. IL-13) to said haempoietic receptor for a time and under conditions sufficient for said treatment to be substantially effected or the conditions to be substantially ameliorated.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as licithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 ug and 2000 mg of active compound.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The present invention also extends to forms suitable for topical application such as creams, lotions and gels.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

The pharmaceutical composition may also comprise genetic molecules such as a vector capable of transfecting target cells where the vector carries a nucleic acid molecule capable of modulating NR4 expression or NR4 activity. The vector may, for example, be a viral vector.

Still another aspect of the present invention is directed to antibodies to NR4 and its derivatives or its ligands (e.g. IL-13). Such antibodies may be monoclonal or polyclonal and may be selected from naturally occurring antibodies to NR4 or may be specifically raised to NR4 or derivatives thereof. In the case of the latter, NR4 or its derivatives may first need to be associated with a carrier molecule. The antibodies and/or recombinant NR4 or its derivatives of the present invention are particularly useful as therapeutic or diagnostic agents.

For example, NR4 and its derivatives can be used to screen for naturally occurring antibodies to NR4. These may occur, for example in some autoimmune diseases. Alternatively, specific antibodies can be used to screen for NR4. Techniques for such assays are well known in the art and include, for example, sandwich assays and ELISA. Knowledge of NR4 levels and/or IL-13 levels may be important for diagnosis of certain cancers or a predisposition to cancers or for monitoring certain therapeutic protocols. In particular, it may be important to monitor an IgE response or levels of IL-13 or IL4 or both which in turn have an effect on the immune system.

Antibodies to NR4 of the present invention may be monoclonal or polyclonal. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool for assessing the receptor or receptor-ligand interaction or monitoring the program of a therapeutic regimin.

For example, specific antibodies can be used to screen for NR4 proteins. The latter would be important, for example, as a means for screening for levels of NR4 in a cell extract or other biological fluid or purifying NR4 made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies or synthetic antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of NR4.

Both polyclonal and monoclonal antibodies are obtainable by immunization with the receptor and either type is utilizable for immunoassays. The methods of obtaining both types of sera are well known in the art. Polyclonal sera are less preferred but are relatively easily prepared by injection of a suitable laboratory animal with an effective amount of NR4, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates a method for detecting NR4 in a biological sample from a subject said method comprising contacting said biological sample with an antibody specific for NR4 or its derivatives or homologues for a time and under conditions sufficient for an antibody-NR4 complex to form, and then detecting said complex.

The presence of NR4 may be accomplished in a number of ways such as by Western blotting and ELISA procedures. A wide range of immunoassay techniques are available as can be seen by reference to U.S. Pat. Nos. 4,016,043, 4, 424,279 and 4,018,653. These, of course, includes both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample is one which might contain NR4 including cell extract, tissue biopsy or possibly serum, saliva, mucosal secretions, lymph, tissue fluid and respiratory fluid. The sample is, therefore, generally a biological sample comprising biological fluid, cell extract, bone marrow or lymph, tissue extract (e.g. from kidney, liver, spleen, etc), fermentation fluid and supernatant fluid such as from a cell culture and cell conditioned medium.

In the typical forward sandwich assay, a first antibody having specificity for the NR4 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable colour change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

Another form of assay involves cells capable of expressing NR4 and L-4 receptor α-chain. For example, if IL4 receptor α-chain and NR4 are co-expressed on cells, such as COS cells, then IL-13 binds to NR4 with a high affinity in the presence of IL-4.

Although not intending to limit the present invention to any one theory or mode of action, when NR4 and the IL-4 receptor are expressed in the same cell, they contribute to the formation of both IL-4 and IL-13 receptors. In the case of IL-4, binding occurs first through the IL-4 receptor α-chain and then NR4 interacts with this complex. In the case of IL-3, binding occurs first to NR4 and then IL-4 receptor α-chain interacts with the complex to form a high affinity receptor capable of signal transduction. The consequences of co-expression of NR4 and IL-4 receptor α-chain is that IL-4 and IL-13 can compete with each other for binding to the IL-4 receptor α-chain and NR4.

Based on this behaviour, it would appear that any protein or small molecule that prevented IL-4 or IL-13 forming cell surface complexes containing both receptor components may be antagonistic. Such molecules may prevent interaction of the cytokine with its low affinity receptor. For example, soluble IL-13BP can prevent IL-13 interaction with NR4. Likewise, soluble IL-4 receptor α-chain can prevent binding of IL-4 to cell surface IL-4 receptor α-chain. These reagents would be antagonists that were specific for IL-4 or IL-13.

By extension, because of its very low affinity, soluble NR4 is a very inefficient IL-13 antagonist. If a soluble NR4 mutant is selected that now binds to IL-4 and also binds to IL-13 with higher affinity, this would be a useful antagonist of both IL4 and IL-13.

An alternative to use of soluble receptor, is to generate a panel of monoclonal antibodies to NR4. If an antibody is obtained which prevents interaction of NR4 with the IL-4 receptor α-chain, a critical event in formation of both functional IL-4 receptor and functional IL-13 receptors, then again the action of both cytokines is inhibited.

In a one particular embodiment the present invention contemplates a method for monitoring the level of IL-4 in a biological sample said method comprising incubating said biological sample with cells which express NR4 and IL-4 receptor α-chain together with an effective amount of IL-13 to competitively inhibit IL-4 binding to its receptor and determining the extent of competitive inhibition.

In a related embodiment the present invention contemplates a method for monitoring the level of IL-13 in a biological sample said method comprising incubating said biological sample with cells which express NR4 and IL-4 receptor α-chain together with an effective amount of IL-4 to competitively inhibit IL-13 binding to its receptor and determining the extent of competitive inhibition.

Preferably, the cytokines are labelled with a reporter molecule as described above.

The biological sample includes but is not limited to blood, serum, plasma, tissue fluid, tissue extract, lymph, T cells or extracts thereof, culture supernatant and conditioned medium.

The present invention also contemplates genetic assays such as involving PCR analysis to detect NR4 gene or its derivatives. Alternative methods or methods used in conjunction include direct nucleotide sequencing or mutation scanning such as single stranded conformation polymorphisms analysis (SSCP) as specific oligonucleotide hybridisation, as methods such as direct protein truncation tests. Such genetic tests may be important, for example, in genetic screening of animals (e.g. humans) for non-expression or substantial absence of expression or expression of mutant forms of NR4 leading to disease conditions.

The nucleic acid molecules of the present invention may be DNA or RNA. When the nucleic acid molecule is in DNA form, it may be genomic DNA or cDNA. RNA forms of the nucleic acid molecules of the present invention are generally mRNA.

Although the nucleic acid molecules of the present invention are generally in isolated form, they may be integrated into or ligated to or otherwise fused or associated with other genetic molecules such as vector molecules and in particular expression vector molecules. Vectors and expression vectors are generally capable of replication and, if applicable, expression in one or both of a prokaryotic cell or a eukaryotic cell. Preferably, prokaryotic cells include E. coli, Bacillus sp and Pseudomonas sp. Preferred eukaryotic cells include yeast, fungal, mammalian and insect cells.

Accordingly, another aspect of the present invention contemplates a genetic construct comprising a vector portion and a mammalian and more particularly a human NR4 gene portion, which NR4 gene portion is capable of encoding an NR4 polypeptide or a functional or immunologically interactive derivative thereof.

Preferably, the NR4 gene portion of the genetic construct is operably linked to a promoter on the vector such that said promoter is capable of directing expression of said NR4 gene portion in an appropriate cell.

In addition, the NR4 gene portion of the genetic construct may comprise all or part of the gene fused to another genetic sequence such as a nucleotide sequence encoding glutathione-S-transferase or part thereof or a cytokine or another haempoietic receptor. Hybrid receptor molecules are particularly useful in the development of multi functional therapeutic and diagnostic agents.

The present invention extends to such genetic constructs and to prokaryotic or eukaryotic cells comprising same.

The present invention also extends to any or all derivatives of NR4 including mutants, part, fragments, portions, homologues and analogues or their encoding genetic sequence including single or multiple nucleotide or amino acid substitutions, additions and/or deletions to the naturally occurring nucleotide or amino acid sequence.

The NR4 and its genetic sequence of the present invention will be useful in the generation of a range of therapeutic and diagnostic reagents and will be especially useful in the detection of a corresponding ligand. For example, recombinant NR4 may be bound or fused to a reporter molecule capable of producing an identifiable signal, contacted with a biological sample putatively containing a ligand and screening for binding of the labelled NR4 to the ligand. Alternatively, labelled NR4 may be used to screen expression libraries of putative ligand genes or functional parts thereof.

In another embodiment, the NR4 is first immobilised. According to this embodiment, there is provided a method comprising contacting a biological sample containing a putative ligand with said haempoietic receptor or a ligand binding portion thereof immobilised to a solid support for a time and under conditions sufficient for a complex to form between said receptor and said ligand if said ligand is present in said biological sample, eluting bound ligand and isolating same.

Soluble NR4 polypeptides as well as various hybrids are also contemplated to be useful in the treatment of disease, injury or abnormality in the nervous system, e.g. in relation to central or peripheral nervous system to treat Cerebral Palsy, trauma induced paralysis, vascular ischaemia associated with stroke, neuronal tumours, motoneurone disease, Parkinson's disease, Huntington's disease, Alzheimer's disease, Multiple Sclerosis, peripheral neuropathies associated with diabetes, heavy metal or alcohol toxicity, renal failure and infectious diseases such as herpes, rubella, measles, chicken pox, HIV or HTLV-1. The NR4 polypeptides and hybrids may also be important for regulating cytokine activity and/or modulating haempoiesis. They are also important for treating allergic or atopic conditions as well as other inflammatory conditions such as rheumatoid arthritis.

As stated above, the NR4 or its ligand of the present invention or their functional derivatives may be provided in a pharmaceutical composition together with one or more pharmaceutically acceptable carriers and/or diluents. In addition, the present invention contemplates a method of treatment comprising the administration of an effective amount of NR4 of the present invention. The present invention also extends to antagonists and agonists of NR4 and/or its ligand and their use in therapeutic compositions and methodologies.

A further aspect of the present invention contemplates the use of NR4 or its functional derivatives in the manufacture of a medicament for the treatment of NR4 mediated conditions defective or deficient.

The present invention is further described by the following non-limiting Figures and Examples.

In the Figures

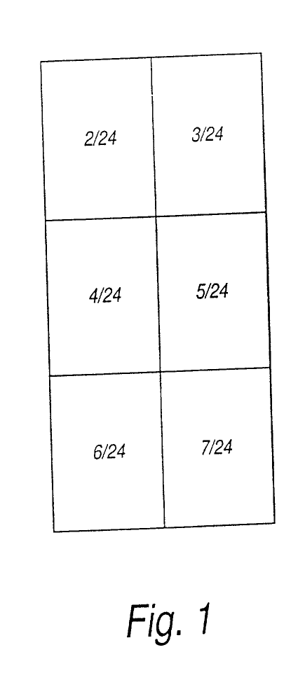

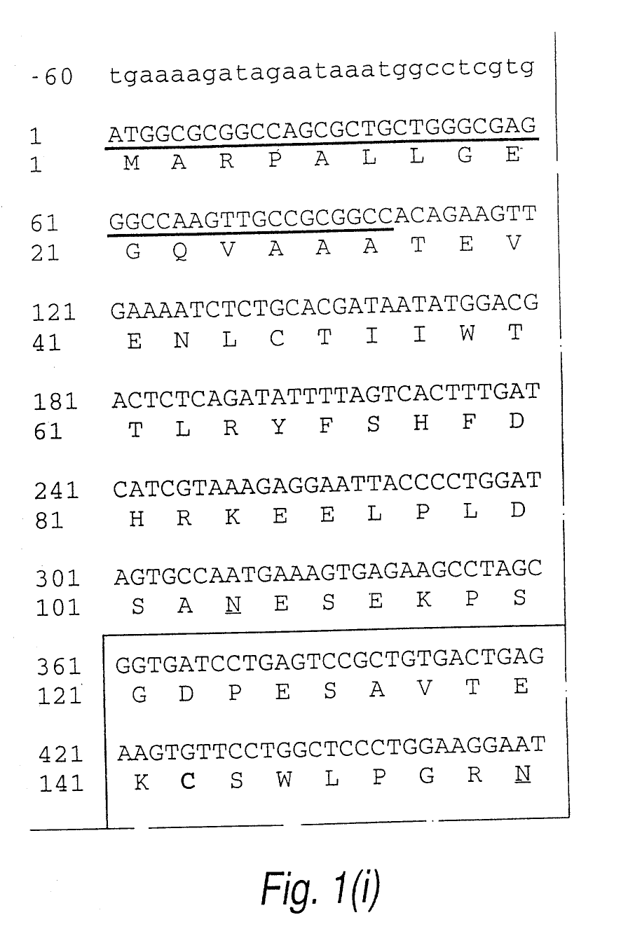

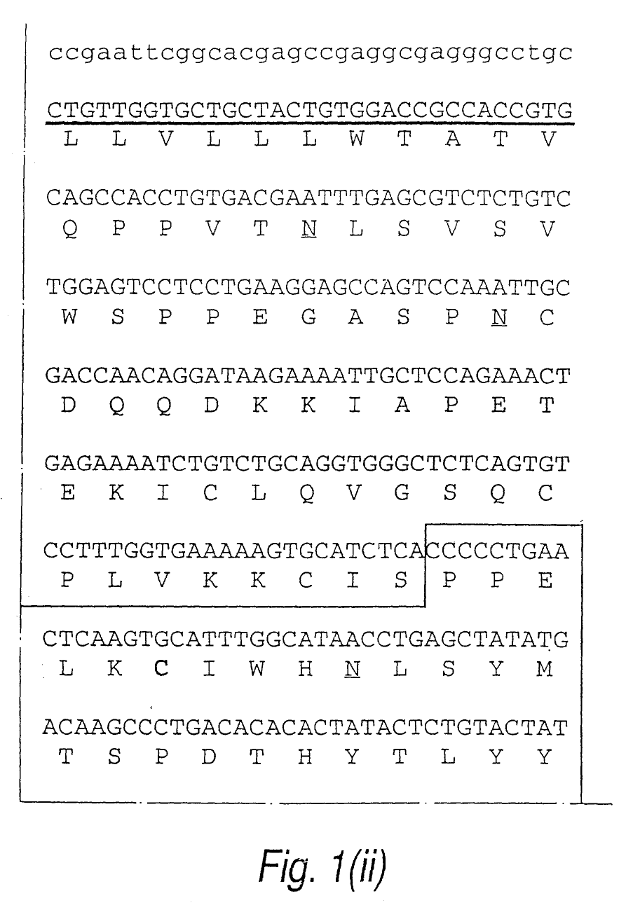

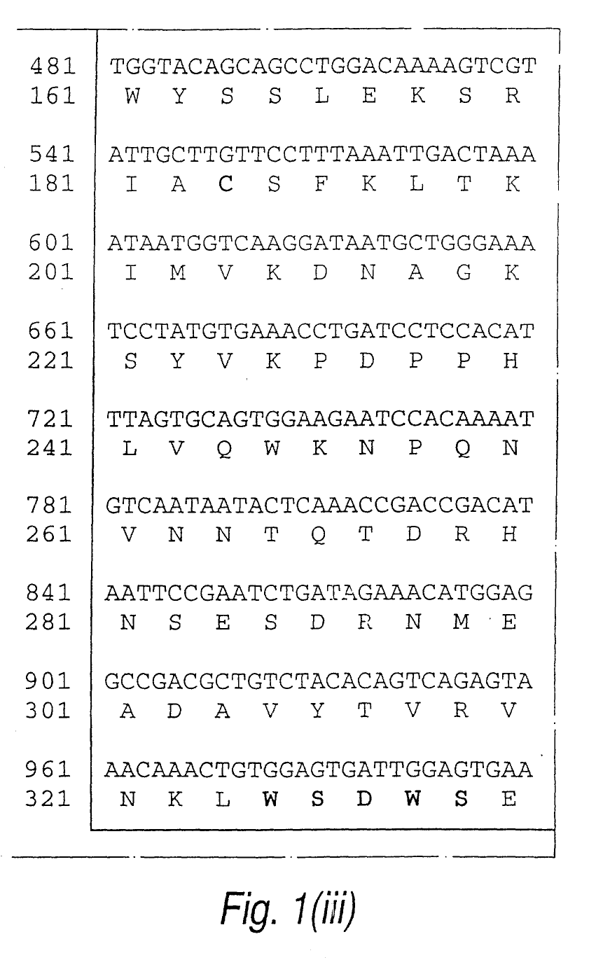

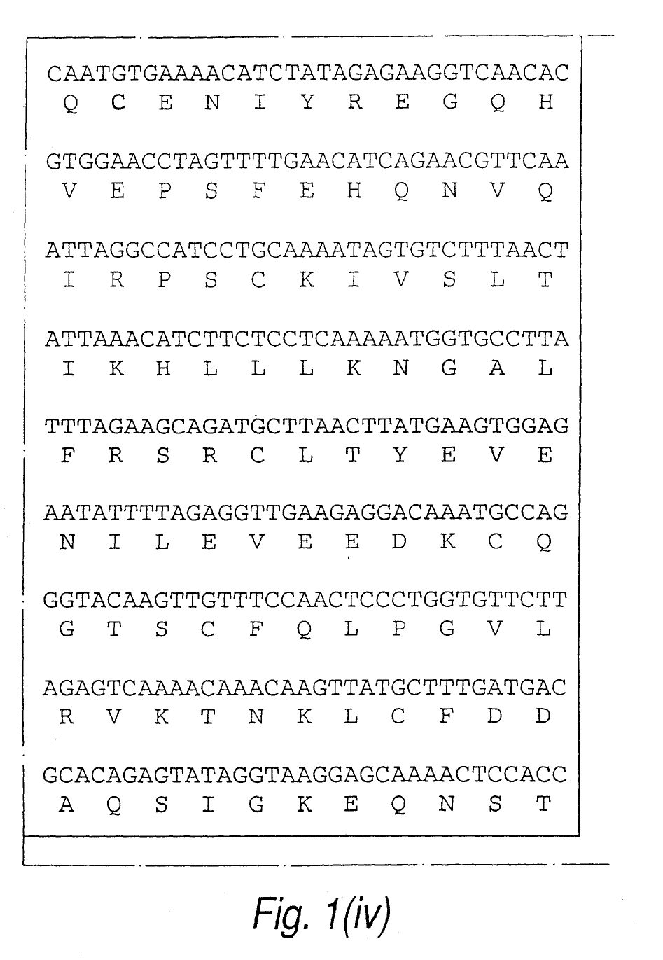

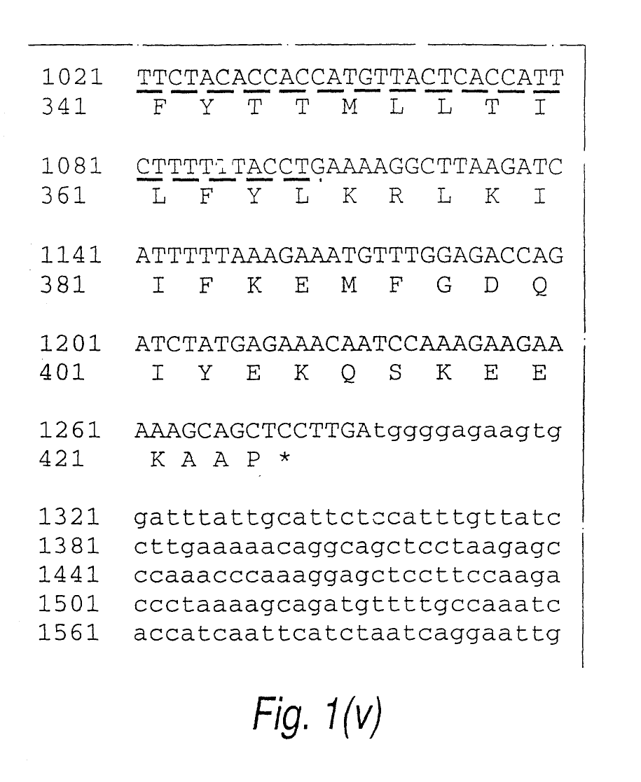

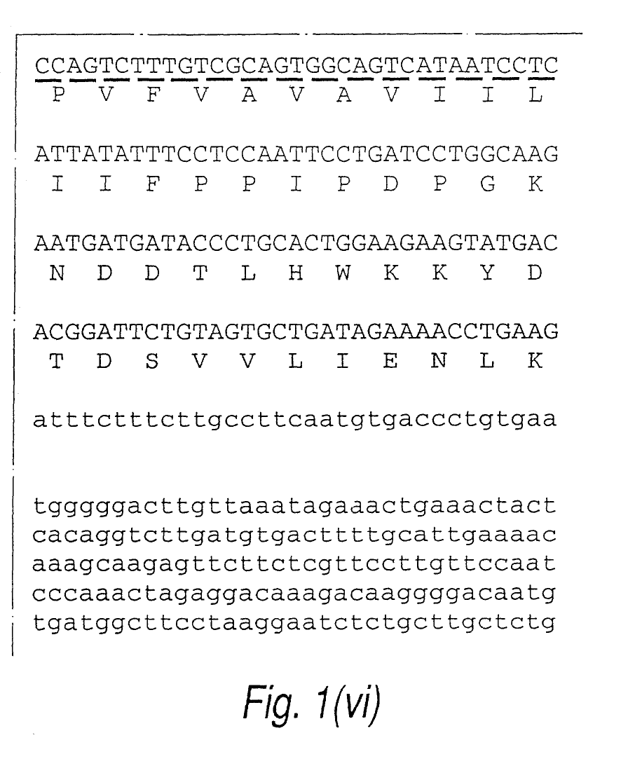

FIG. 1 is a representation of the nucleotide [SEQ ID NO:1] and predicted amino acid [SEQ ID NO:2] sequence of murine NR4. The untranslated region is shown in lower case and the translated region in upper case. The conventional one-letter code for amino acids is employed, potential asparagine linked glycosylation sites are underlined and the conserved cysteine residues and WSXWS motif of haempoietin receptor family members are shown in bold. The predicted signal sequence is underlined in bold while the transmembrane domain is underlined with dashes. The sequence shown is a composite derived from the analysis of 8 cDNA clones derived from 3 libraries. The 5′−end of the sequence (nucleotides −60 to 351) is derived from a single cDNA clone but is also present in genomic DNA clones that have been isolated. Boxed region-typical haempoietin receptor domain, amino acids 118-340.

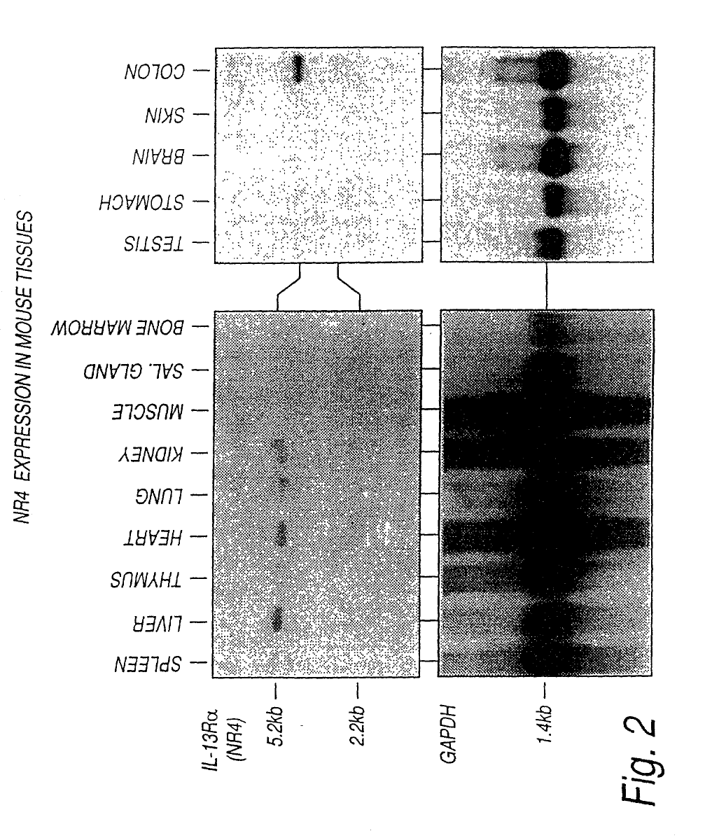

FIG. 2 is a photographic representation showing northern analysis of murine NR4 mRNA expression in selected tissues and organs.

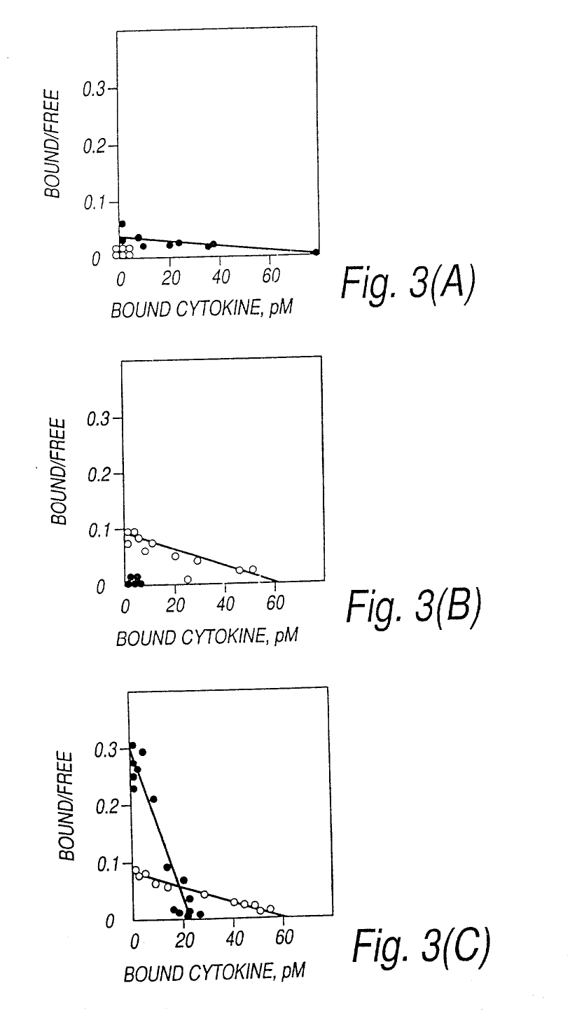

FIG. 3 is a graphical representation depicting saturation isotherms of ¹²⁵I-IL-13 and ¹²⁵I-IL-4 binding; saturation isotherms depicted as Scatchard plots of IL-4 (°) and IL-13 (•) binding to (A) COS cells expressing the IL-13Rα(NR4), (B) CTLL cells and (C) CTLL cells expressing the IL-13Rα(NR4). Data have been normalised to 1×10⁴ COS cells and 1×10⁶ CTLL cells and binding was carried out on ice for 2 to 4 hours.

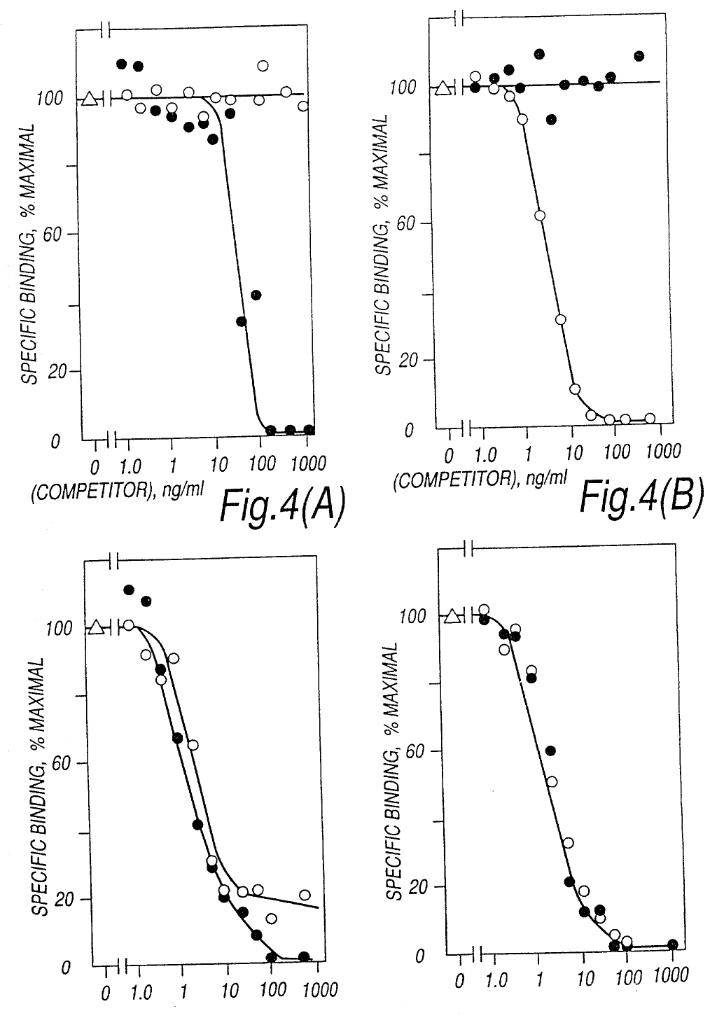

FIG. 4 is a graphical representation showing specificity of IL-4 and IL-13 binding; the ability of IL-4 (°) and IL-13 (•) to compete for ¹²⁵I-IL-13 binding to (A) COS cells expressing the IL-13Rα(NR4) and (C) CTLL cells expressing the IL-13Rα(NR4) or to compete for ¹²⁵I-IL-4 binding to (B) CTLL cells and (D) CTLL cells expressing the IL-13Rα(NR4). Binding was carried out at 4° C. for 2 to 4 hours and the data expressed as a percentage of the specific binding observed in the absence of a competitor (Δ).

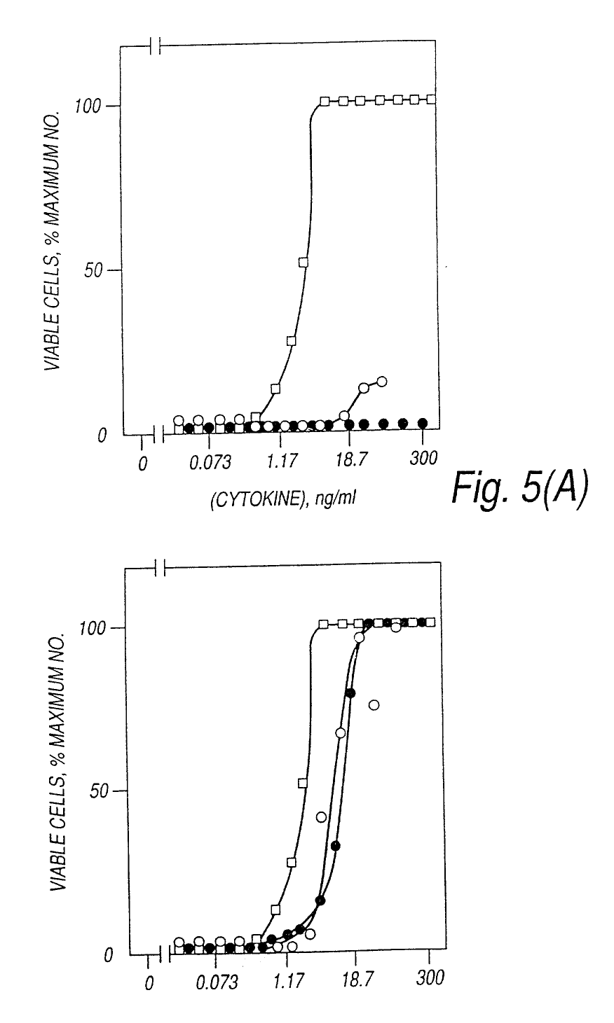

FIG. 5 is a graphical representation showing factor dependent proliferation of cells expressing NR4. Two hundred (A) CTLL cells or (B) CTLL cells expressing the IL-13Rα(NR4) were incubated in the absence of cytokine or with various concentrations of IL-2 (□), IL-4 (□) or IL-13 (•). After 48 hours viable cells were counted and data were expressed as a percentage of the number of viable cells observed with a maximal concentration of IL-2.

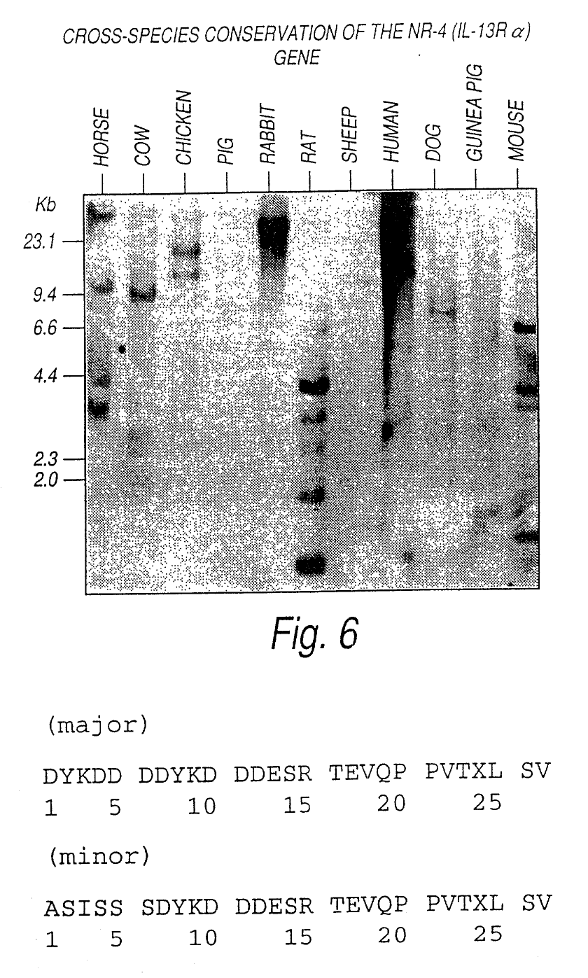

FIG. 6 is a photographic representation showing cross-species conservation of NR4 (IL-13Rαgene.

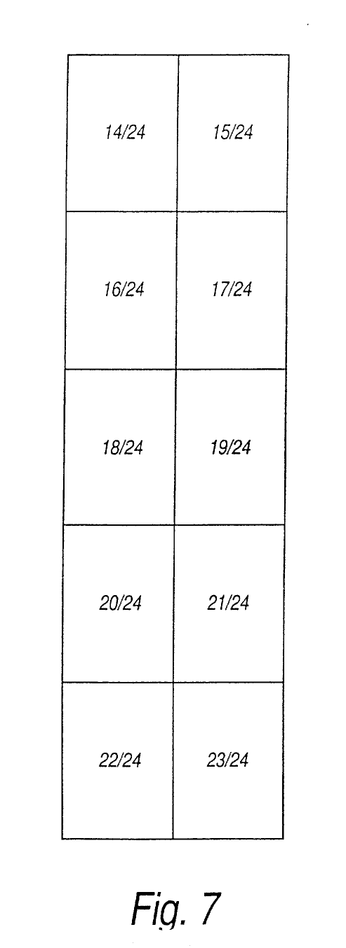

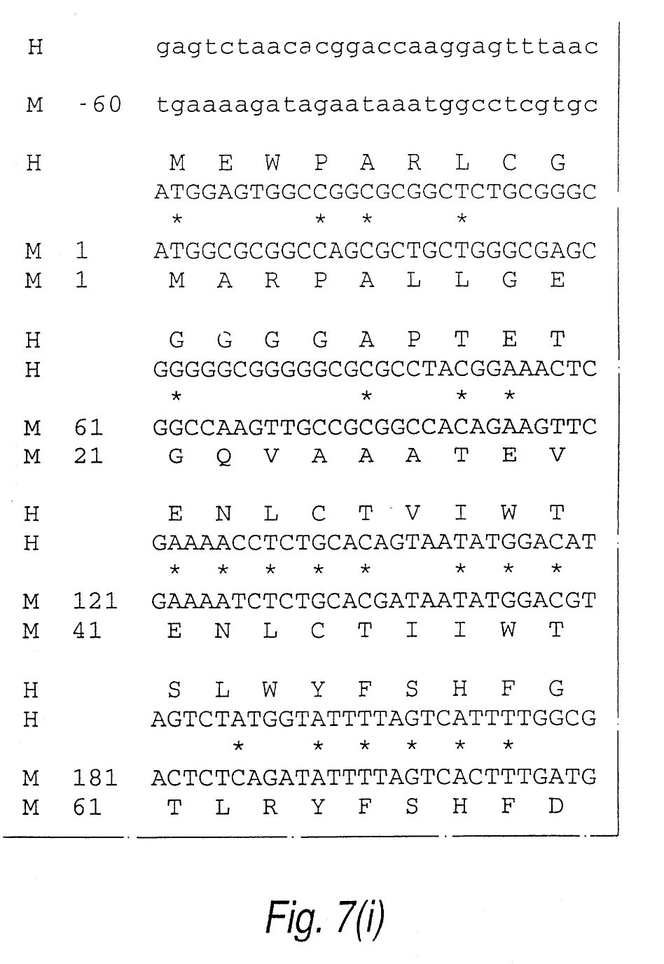

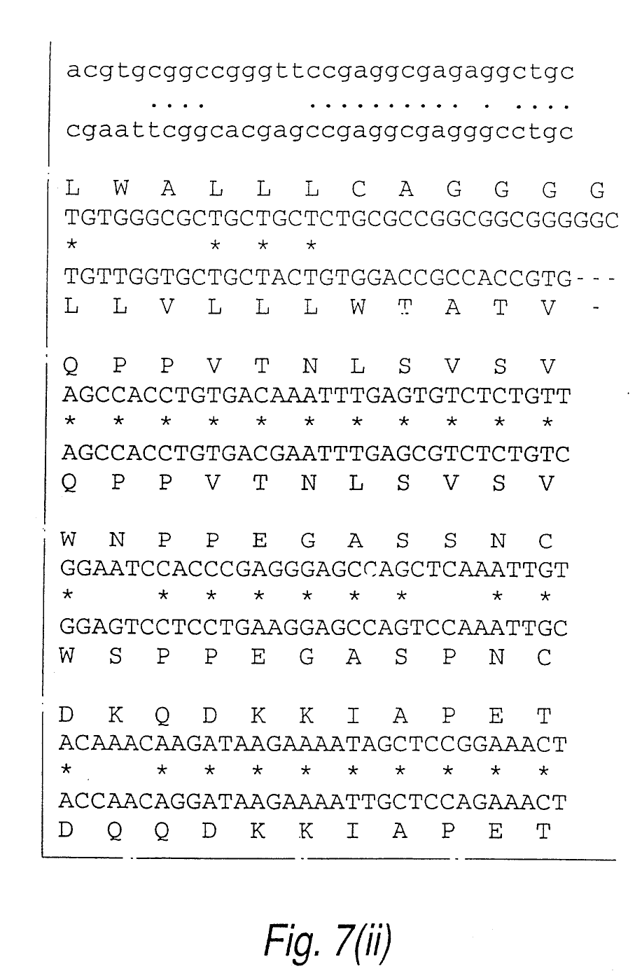

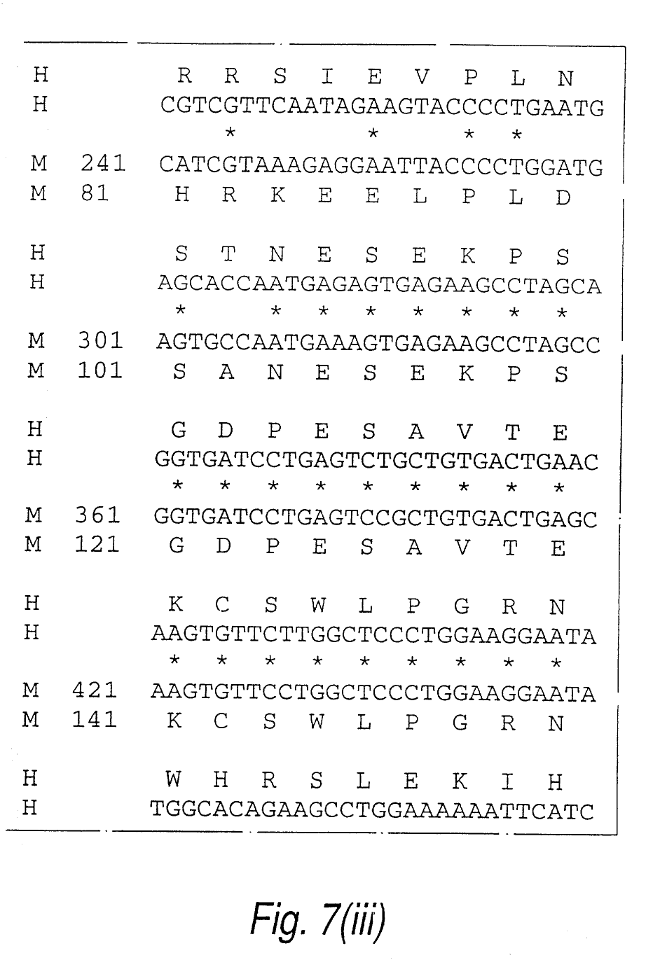

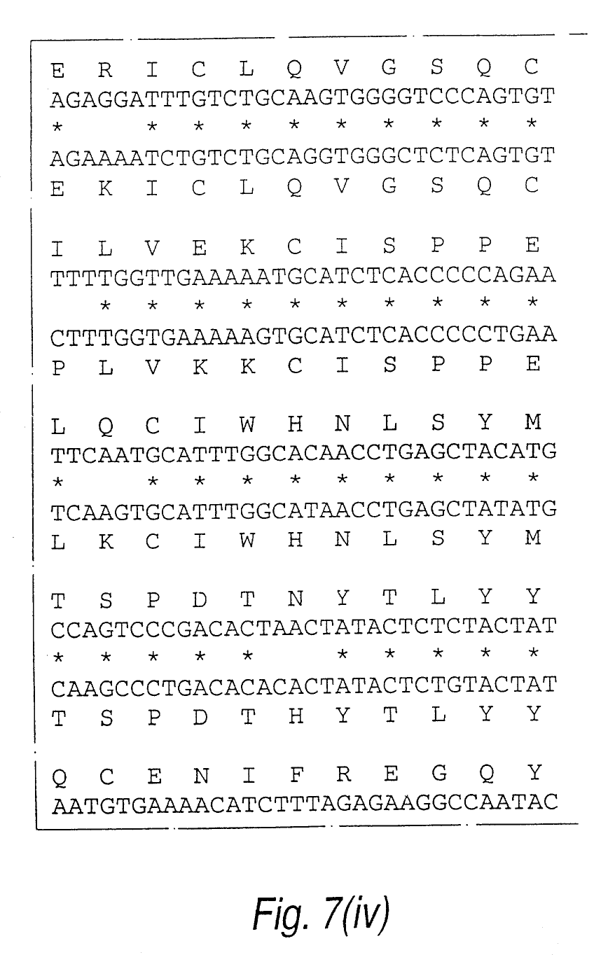

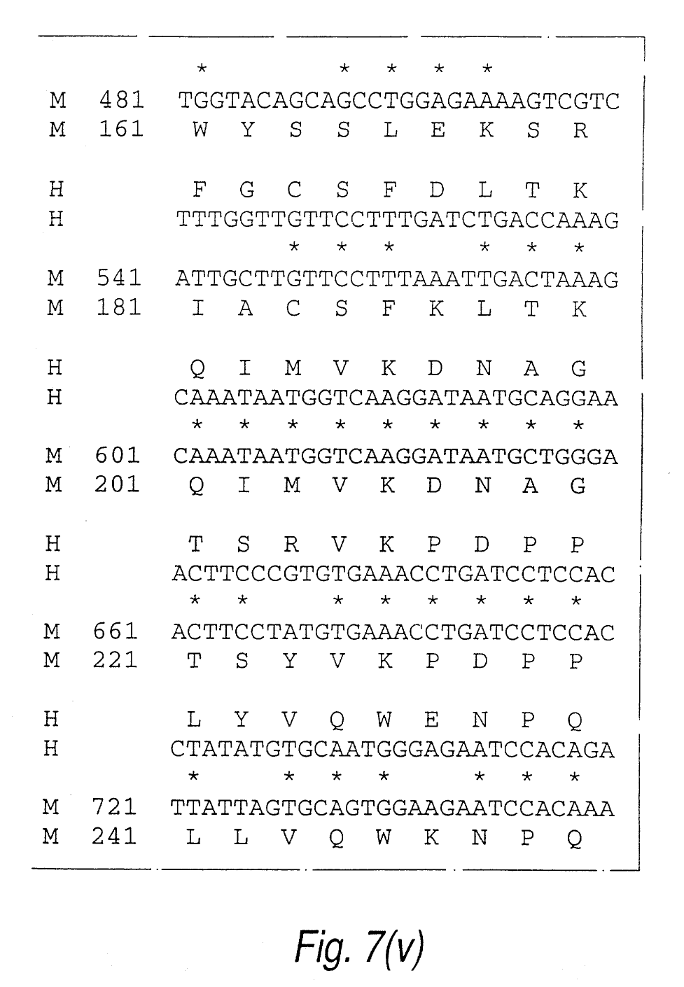

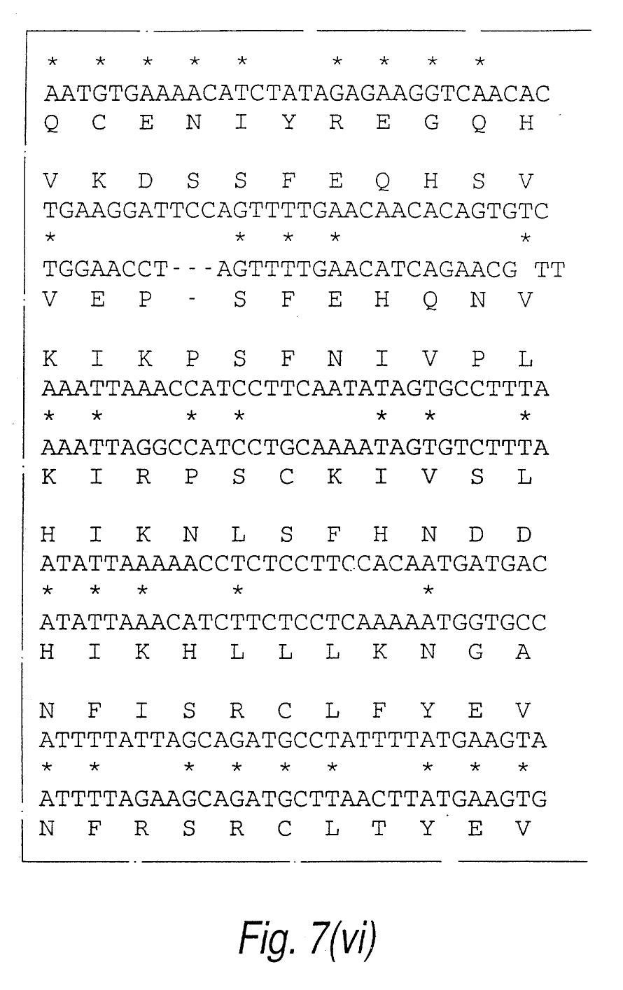

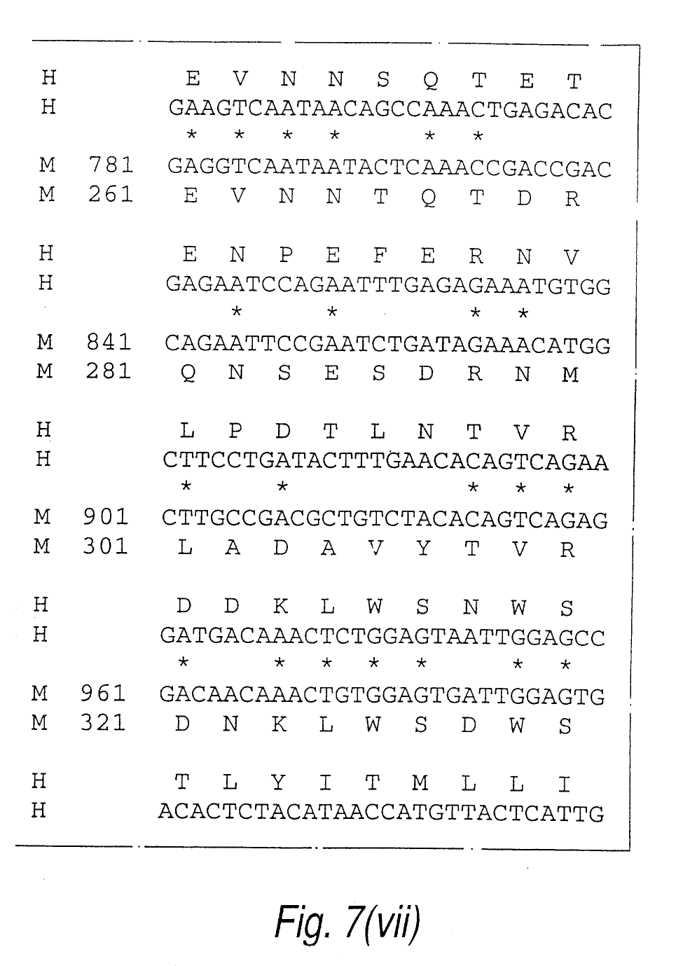

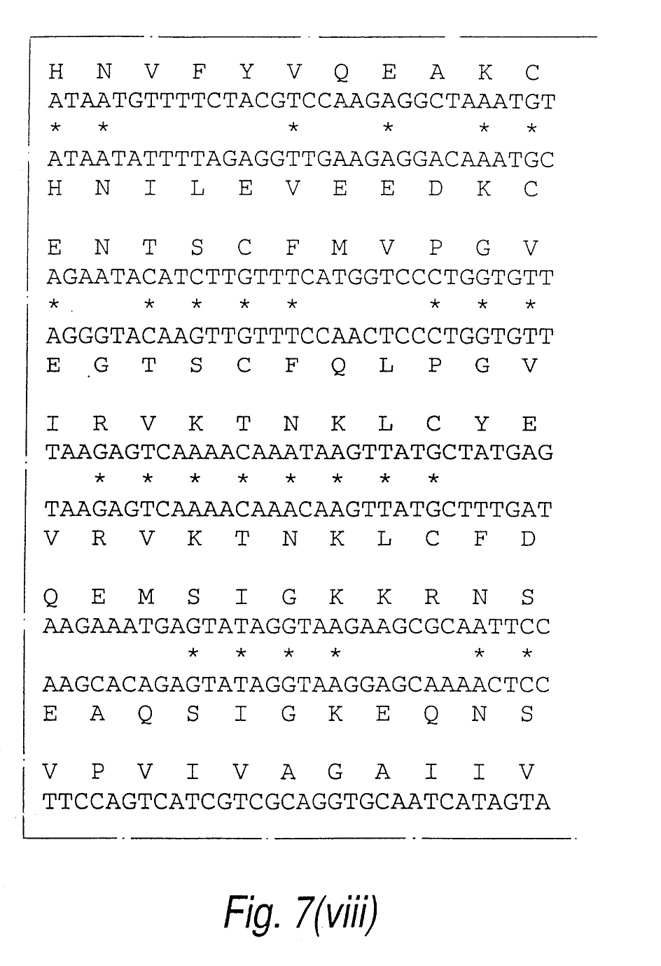

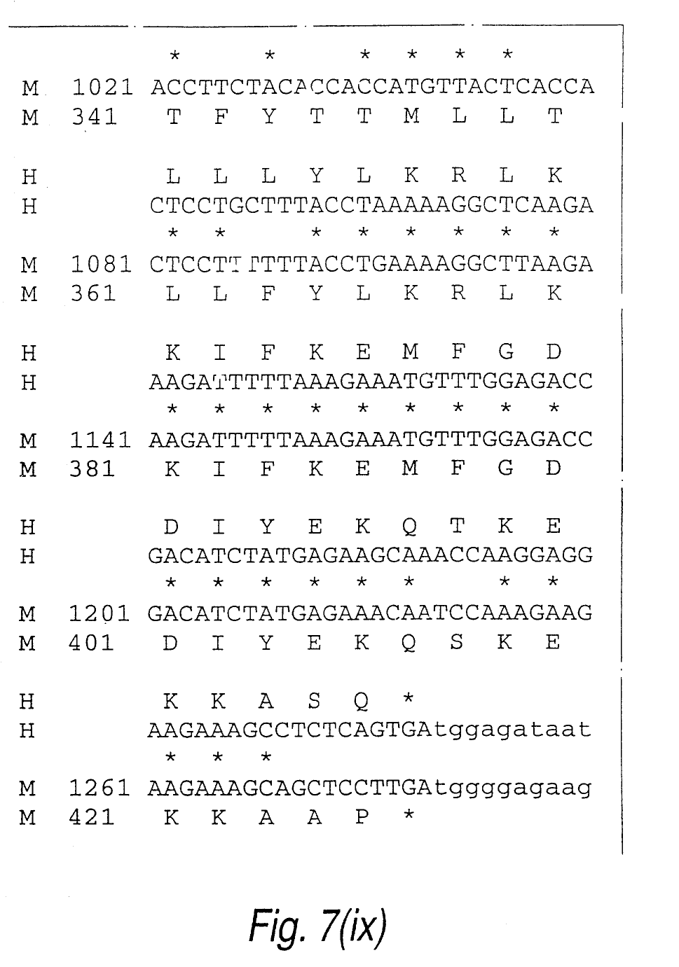

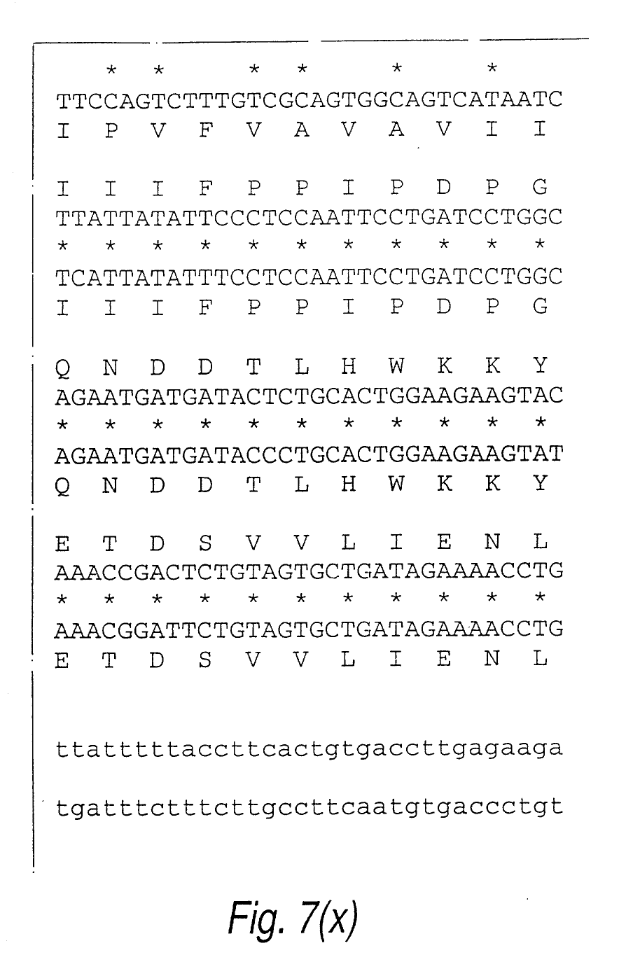

FIG. 7 is a representation of the nucleotide and corresponding amino acid sequence of murine and human NR4 (IL-13Rα) genes. The nucleotide and predicted amino acid sequence of human (H) and murine (M) IL-13Rα(NR4) were aligned by eye, with gaps (−) inserted to optimise the alignment. The numbering is for the murine clone, nucleotides that form part of the coding region are shown in upper case, whilst those of the untranslated regions are shown in lower case. Amino acids identical between the predicted murine and human proteins are indicated by (*). DNA encoding the murine signal sequence is underlined, with A26 or T27 being the predicted first amino acid of the mature protein.

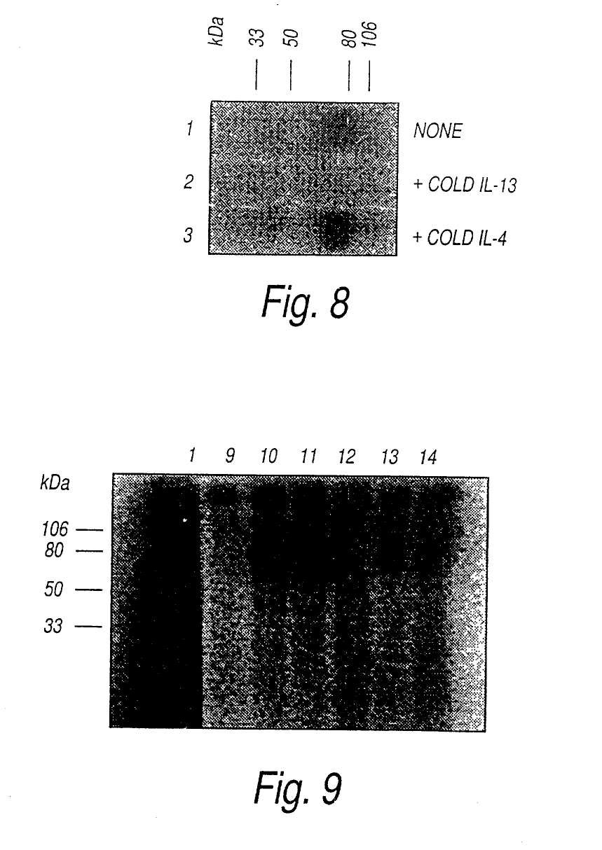

FIG. 8 is a photographic representation showing ¹²⁵I-IL-13 cross-linking to soluble NR4. Lane: ¹²⁵I-IL13 (100,000 cpm)+2μg/ml soluble NR4; Lane 2: ¹²⁵I-IL-13 (100,000 cpm)+2μg/ml soluble NR4 in the presence of excess unlabelled IL-13; Lane 3: ¹²⁵I-IL-13 (100,000 cpm)+2μg/ml soluble NR4 in the presence of excess unlabelled IL-4.

FIG. 9 is a photographic representation of immunoprecipitation by anti-NR4 polyclonal antisera of cross-linked ¹²⁵I-IL-13 with IL-13Rα(NR4). Lanes 9-11: soluble IL-13Rα(30 μl of 3 μg/ml) cross-linked to ¹²⁵IL-13 (750,000 cpm) and immunoprecipitated with control rabbit serum, or with anti-NR4 polyclonal antiserum in the presence or absence of 100 μg/ml FLAG peptide, respectively; Lanes 12-14: soluble IL-13Rα(NR4) (30 μl of 3 μg/ml) cross-linked to ¹²⁵I-IL-13 (750,000 cpm) in the presence of 0.5 μg/ml unlabelled IL-13 and immunoprecipitated with an anti-IL-13Rα(NR4) polyclonal antiserum also in the presence or absence of 100 μg/ml FLAG peptide, respectively.

FIG. 10 is a representation of the N-terminal amino acid sequence of murine NR4.

The following single and three letter abbreviations for amino acid residues are used in the specification:

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### EXAMPLE 1

### Isolation of genomic and cDNAs encoding NR4

ApoI digested genomic DNA, extracted from an embryonal stem cell line, was cloned into the λZAPII bacteriophage (Stratagene, LaJolla, Calif. ). Approximately 10⁶ plaques from this library were screened with a ³²P-labelled oligonucleotide corresponding to the sequence Trp-Ser-Asp-Trp-Ser (16). Positively hybridising clones were sequenced using an automated DNA sequencer according to the manufacturer's instructions (Applied Biosystems, Foster City, Calif.). One clone appeared to encode for part of a new member of the haemopoietin receptor family. Oligonucleotides were designed on the basis of this genomic DNA sequence and were used in the conventional manner to isolate clones from mouse peritoneal macrophage (Clontech Laboratories, Palo Alto, Calif.), mouse skin, mouse lung, mouse kidney, and WEHI-3B., (Stratagene, LaJolla, Calif.) λ-bacteriophage cDNA libraries.

### EXAMPLE 2

### Construction of expression vectors and transfection of cells

Using PCR, a derivative of the NR4 cDNA was generated which encoded for the IL-3 signal sequence [SEQ ID NO:5] and an N-terminal FLAG epitope-tag [SEQ ID NO:6] preceding the mature coding region of NR4 (Thr27 to Pro424; FIG. 1). The PCR product was cloned into the mammalian expression vector pEF-BOS (17). Constructs were sequenced in their entirety prior to use. Cells were transfected and selected as previously described (16, 18).

### EXAMPLE 3

### Northern blots

Northern blots were performed as previously described (16). The source of hybridisation probes was as follows: NR4-a PCR product from nucleotide 32 to 984 (FIG. 1) and GAPDH-a cDNA fragment spanning nucleotides (19).

### EXAMPLE 4

### Cytokines and experiments using radioiodinated cytokines

IL-2, IL4, IL-7, IL-9, IL-13 and IL-15 were obtained commercially (R & D Systems, Minneapolis Minn.). For radioiodination, cytokines were dissolved at a concentration of 100 μg/ml in 10 mM sodium phosphate, 150 mM NaCl (PBS), 0.02% v/v Tween 20 and 0.02% w/v sodium azide at pH 7.4. An amount of 2μg of IL-13 was radioiodinated using the iodine monochloride method (20, 21), while 2μg of IL-4 was radiolabelled using di-iodo-Bolton-Hunter Hunter reagent (16). Binding studies and determination of the specific radioactivity and bindability of labelled cytokines were performed as previously described (2).

For cross-linking experiments, recombinant murine IL-13 was produced as a FLAG-tagged protein in Pichia pastoris.

For cross-linking assays, aliquots of purified soluble IL-13Rα(NR4) were incubated with ¹²⁵I-IL-13 in the presence or absence of a competitor in a final volume of 20 μl for at least 30 min at 40° C. Then 5 μl of a 12 mM solution of BS³ (Bis (Sulfosuccimidyl) suberate) in PBS containing 0.02% v/v Tween-20 was added and the mixtures were incubated for 30 min at 4° C. Samples were mixed with 8 μl of 4XSDS sample buffer and analysed by 13% w/v SDS-PAGE under non-reducing conditions. Gels were dried and visualised by either autoradiography or with a PhosphoImager.

### EXAMPLE 5

### Proliferation Assays

The proliferation of Ba/F3 and CTLL cells in response to cytokines was measured in Lux 60 microwell HL-A plates (Nunc Inc. Ill., USA). Cells were washed three times in DMEM containing 20% v/v new born calf serum and resuspended at a concentration of 2×10⁴ cells per ml in the same medium. Aliquots of 10μl of the cell suspension were placed in the culture wells with 5 μl of various concentrations of purified recombinant cytokines. After 2 days of incubation at 37° C. in a fully humidified incubator containing 10% V/V CO₂ in air, viable cells were counted using an inverted microscope.

### EXAMPLE 6

### Cloning and Characterisation of Murine NR4

A library was constructed in λZAP II using ApoI digested genomic DNA from embryonal stem cells and screened with a pool of ³²P-labelled oligonucleotides encoding the amino acid sequence Trp-Ser-Asp-Trp-Ser found in many members of the haemopoietin receptor family. One hybridising bacteriophage clone was found to contain a sequence that appeared to encode part of a novel member of the haemopoietin receptor family. This receptor was given the operational name NR4 The sequence of the genomic clone was used to isolate cDNAs encoding NR4 from WEHI-3B cell, peritoneal macrophage, bone marrow, skin and kidney libraries. A composite of the nucleotide sequence [SEQ ID NO:1] and predicted amino acid sequence [SEQ ID NO:2] of these cDNAs is shown in FIG. 1. The NR4 cDNA is predicted to encode for a protein of 424 amino acid residues, containing a putative signal sequence and transmembrane domain. The extracellular region of the protein contained an immunoglobulin-like domain (amino acids 27-117), in addition to a typical haemopoietin receptor domain (amino acids 118-340) which includes four conserved cysteine residues and the characteristic Trp-Ser-Asp-Trp-Ser motif (FIG. 1; in bold as WSXWS). The cytoplasmic tail of the new receptor was 60 amino acids in length.

### EXAMPLE 7

### Expression pattern of NR4 cDNA

The pattern of NR4 mRNA expression was examined by Northern analyses. Two hybridising species of 5.2 and 2.2 kb in length were detected in mRNA from most tissues (FIG. 2). NR4 mRNA was not detectable in skeletal muscle (FIG. 2). FIG. 8 shows expression of NR4 in mouse tissues.

### EXAMPLE 8

### NR4 encodes the IL 13 receptor α-chain (IL-13Rα)—a specific binding subunit of the IL-13 receptor

The apparent molecular mass is from about 50,000 to about 70,000 daltons and more particularly about 55,000 to about 65,000 daltons for NR4 expressed in COS cells estimated from Western blots using an anti-FLAG antibody. This suggested that NR4 might encode the binding subunit of the IL-13 receptor in order to test this possibility, NR4 was expressed in COS cells. Untransfected COS cells expressed relatively low levels of IL-4 and IL-13 receptors. Upon transfection with a plasmid containing the NR4 cDNA, the number of IL-13 receptors but not IL-4 receptors expressed by COS cells was dramatically increased (FIG. 3A; 100,000 to 500,000 receptors per cell). The affinity of IL-13 for NR4 expressed by COS cells was low (K_{D}˜2-10 nM) and binding was specific since it could compete with unlabelled IL-13 (FIG. 4A) but not other cytokines including IL-2, IL-4, IL-7, IL-9 or IL-15. These results suggest that NR4 is the IL-13 receptor α-chain (IL-13Rα).

### EXAMPLE 9

### The IL-13Rα(NR4) and the IL-4Rαare shared components of the IL-4 and IL-13 receptors

In order to investigate the relationship between IL-4 and IL-13 receptors, the IL-4 responsive cell line CTLL was examined. Parental CTLL cells expressed a single class of IL-4 receptor (K_{D}˜660 pM; ˜3600 receptors per cell) but no detectable IL-13 receptors (FIG. 3B). The IL-4 receptors expressed by CTLL cells appeared to be specific since binding of ¹²⁵-IL-4 could compete with unlabelled IL-4 but not IL-13 (FIG. 4B). Upon expression of the IL-13 Rα(NR4) in CTLL cells no change was observed in the number or affinity of IL-4 receptors, while a single class of high affinity IL-13 receptors was detected (FIG. 3C; K_{D}˜75 pM; 1350 receptors per cell). The affinity of IL-13 for the IL-13Rα(NR4) expressed in CTLL cells was higher than in COS cells, suggesting that the former expressed a protein capable of interacting with the IL-13Rα(NR4) to increase the affinity for IL-13. A likely candidate based on previous studies is the IL-4Rα. In order to explore this possibility the ability of IL-4 to compete with ¹²⁵I-IL-13 for binding to CTLL cells expressing the IL-13Rα(NR4) was assessed. FIG. 4B shows that IL-4 and IL-13 were equally effective in competing for ²⁵I-IL-13 binding (IC₅₀˜300pM; FIG. 4C) and, in addition, were able to compete with ¹²⁵I-IL-4 for binding (IC₅₀˜300 pM; FIG. 4D).

### EXAMPLE 10

### Expression of the IL-13Rα(NR4) is necessary for transduction of a proliferative signal by IL-13

CTLL cells require the addition of exogenous cytokines for survival and proliferation. IL-2 was found to be a potent proliferative stimulus for CTLL cells (EC₅₀˜100-200 pM), while IL-4 was relatively weak (EC₅₀2-7 nM) and IL-13 was inactive (FIG. 5A). Expression of the IL-13Rα(NR4) in CTLL cells resulted in the ability to survive and proliferate weakly in, response to IL-13 (EC₅₀˜700 pM) and to proliferate somewhat more strongly than parental cells in response to IL-4 (EC₅₀˜700 pM; FIG. 5B).

### EXAMPLE 11

### Cloning of Human IL 13Rα(NR4)

In order to determine whether genes homologous to murine IL-13Rα(NR4) exist in other vertebrate species, a probe encompassing nucleotides 840 to 1270 of murine IL-13Rα(NR4) was hybridised to EcoRI digested genomic DNA from various species. Hybridisation was carried out in 500 mM Na₂HPO₄ (˜5×SSC) at 50° C. overnight. The filter was washed in 40 mM Na₂HPO₄ (˜0.2×SSC) at 50° C. for 2 hours and exposed to autoradiographic film for 48 hours. FIG. 6 illustrates that relatively few (1 to 5) hybridising bands are observed in genomic DNA from various species, including human. This suggests that it is feasible to clone human IL-13Rα(NR4) using a murine cDNA probe. A human bone marrow cDNA library clones in the λZAPII bacteriophage was therefore screened with two probes (nucleotides 82-840 and 840 to 1270) from the murine IL-13Rα(NR4) cDNA. Hybridisation was carried out overnight in 6×SSC, 0.1% w/v SDS at 42° C. Filters were washed at 2×SSC, 0.1% w/v SDS at 50° C. for 2 hours and exposed for 48 hours to autoradiographic film. Plaques that hybridised to both murine IL-13Rα(NR4) probes were picked and purified in the conventional manner. The cDNA inserts form the hybridising bacteriophage were excised into the pBluescript plasmid and sequenced in their entirety using an ABI automated sequencer. FIG. 7 shows a composite of the sequence of the clones isolated and reveals that the clones encode a protein that shares a high degree of sequence similarity with murine IL-13Rα(NR4). The clones encode the entire coding region of the protein. The high degree of sequence similarity (320/425 amino acids ˜75%) predicates that this cDNA is the human homologue of the murine IL-13Rα(NR4). The nucleotide sequence is represented as SEQ ID NO:3 and the amino acid sequence is SEQ ID NO:4.

### EXAMPLE 12

### Soluble Murine IL-13Rα(NR4) binds IL-13

Constructs were engineered to express soluble versions of NR4 with an N-terminal “FLAG” epitope (International Biotechnologies/Eastman Kodak, New Haven Conn.). First, a derivative of the mammalian expression vector pEF-BOS was generated so that it contained DNA encoding the signal sequence of murine IL-3 (MVLASSTTSIHTMLLLLLMLFHLGLQASIS [SEQ ID NO:5]) and the FLAG epitope (DYKDDDDK [SEQ ID NO:6]), followed by a unique XbaI cloning site. This vector was named pEF/IL3SIG/FLAG. The mature extracellular part of the NR4 coding region (Thr27 to Thr344) was generated by PCR using primers 1478 and 1480. The resulting product was digested with XbaI and was cloned into the XbaI site of pEF/IL3SIG/FLAG to give pEF/IL3SIG/FLAG/sol NR4. The identity of the construct was confirmed by dideoxy sequencing. OLIGO 1478 5′ AGCTTCTAGAACAGAAGTTCAGCCACCTGTG 3′ [SEQ ID NO:7]; OLIGO 1480 5′ AACTCCACCTTCTACACCACCTGATCTAGA 3′ [SEQ ID NO:8].

After transfection into CHO cells, expressed, soluble NR4 was purified from CHO cell-conditioned medium on an anti-FLAG antibody (M2) affinity column by elution with free FLAG peptide (Science Imaging Systems).

Consistent with the low affinity of IL-13 for NR4 expressed by COS cells, purified soluble NR4 appeared unable to bind IL-13 as assessed by gel filtration chromatography. However, using sensitive cross-linking assays, the ability of soluble IL-13Rα(NR4) to bind IL-13 was demonstrated (FIG. 8, lane 1). This interaction was competed for by unlabelled IL-13 but not by unlabelled IL-4 (FIG. 8, lanes 2 and 3).

### EXAMPLE 13

### A Polyclonal Antisera to Soluble IL13Rα(NR4)

A polyclonal antiserum to NR4 was prepared by injecting purified soluble NR4 into rabbits which were bled after 3 months. This antisera immunoprecipitated the cross-linked product of ¹²⁵I-IL-13 with soluble NR4 (FIG. 9, lane 11) while no immunoprecipitation was observed with pre-immune serum (FIG. 9, lane 9). Immunoprecipitation of the complex was not inhibited by the FLAG peptide (FIG. 9, lane 10).

The immunoprecipitation assay was conducted as follows:

The cross-linking reactions were terminated by the addition of Tris-HCl, pH 7.5, to a final concentration of 40 mM. The samples were then mixed with 1:50 diluted control rabbit serum or anti-NR4 serum which had been pre-incubated with or without FLAG peptide. After incubation for 30 min at 4° C., the mixtures were added to 40 μl of 50% v/v protein G-Sepharose Sepharose gel slurry (Pharmacia) and incubated for 30 min at 4° C. The samples were centrifuged and the protein G beads were washed 3×0.5 ml PBS, mixed with 40 μl of 2×concentrated SDS-PAGE sample buffer and heated for 2 min at 95° C. The supernatants were analysed by 13% w/v SDS-PAGE under non-reducing conditions.

### EXAMPLE 14

### N-terminal Amino Acid Sequence of NR4

The N-terminal amino acid sequence of NR4 was determined and is shown in FIG. 10.

### EXAMPLE 15

### Assay for IL-13

IL-13 is a cytokine that is implicated in the production of IgE, the immunoglobulin isotype important in allergic diseases such as asthma. Monitoring IL-13 levels may, therefore, be an important diagnostic. Since IL-4 and IL-13 share many biological effects, generating an assay that discriminates these cytokines is also important.

NR4 expressed in COS cells binds ¹²⁵I-IL-13. This binding is inhibited in a dose dependent manner by unlabelled IL-13, in the presence of a large amount of irrelevant protein such as calf serum or human serum. IL-4 shows no ability to compete for ¹²⁵I-IL-13 binding in this situation and, therefore, this assay appears to be specific for IL-13.

The assay is set up by coating soluble NR4 on ELISA plates and using, for example, fluorescent labelled IL-13 as the probe. The presence of unlabelled IL-13 in a test sample then registers as a decrease in the fluorescent signal.

Similar assays are set up that measure both IL-4 and IL-13 by using cells that express NR4 and IL-4 receptor α-chain. These include CTLL cells which normally express IL-4 receptor α-chain and which are engineered to express NR4. Binding of ¹²⁵I-IL-13 or ¹²⁵I-IL-4 can be inhibited by unlabelled forms of both IL-4 and IL-13.

### EXAMPLE 16

### Modifications to IL-4 and IL-13

Mutations are introduced into regions of the molecules that are predicated to be functionally important. In the case of NR4, this includes the region that interacts with IL-13, the region which interacts with IL-4 receptor α-chain or the region that interacts with IL-4 when this cytokine is bound to the IL-4 receptor α-chain. These regions are determined by direct experiment, for example, by solving the structure of NR4 or complexes of NR4 with other proteins like IL-4, IL-13 and the IL-4 receptor α-chain or by modeling these proteins on similar proteins for which structural information exists, for example, the growth hormone/growth hormone receptor complex. Resulting NR4 mutants are then individually tested for improved function.

In an alternative method, random mutations are generated in the molecules. Suitable techniques include synthesis of NR4 cDNA using a polymerase and reaction conditions that promote incorporation of the incorrect dNTP and use of a technique called DNA shuff mg (23, 24, 25, 26).

After generating random mutants of the cDNA of interest, potentially useful mutants are selected. In the case of NR4, an assay is based on knowledge that if NR4 is expressed in cells which lack IL-4 receptor α-chain (e.g. COS cells), then cells are obtained that cannot bind IL-4 with any detectable affinity and binds IL-13 with low affinity. Thus, if COS cells are transfected with Nr4 and allowed to bind FITC-conjugated IL-4 and phycoerythrin-conjugated IL-13, the unbound ligand washed away, the no IL-4 will bind and any IL-13 that had bound would dissociate during the washing.

If these cells are FACS-sorted, then little or no signal in either the FITC or PE channel would be obtained. COS cells are transfected with 10⁶ to 10⁷ random mutants of NR4 and processed for binding. Any cells sorted which bind the cytokines better than those transfected with wild type NR4 can be FACS sorted. The plasmids containing these “improved” NR4 cDNAs may be recovered, expanded in E. coli and used again in COS cells to confirm the improvement. Any mutants that are consistently better can then be used for the introduction of further random changes into an order to get even better molecules. This iterative process may be repeated several times.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. An isolated nucleic acid molecule comprising a sequence of nucleotides encoding or complementary to a sequence encoding an haemopoietin receptor from an animal or a derivative of said receptor.

2. An isolated nucleic acid molecule comprising a sequence of nucleotides encoding or complementary to a sequence encoding an animal haempoietin receptor or a derivative thereof, wherein said receptor:
(i) is capable of interaction with IL-13 or its derivatives; and
(ii) is capable of interaction with a complex between IL-4 and IL-4 receptor α-chain.

3. An isolated nucleic acid molecule according to claim 1 or 2 wherein the receptor comprises a derivative of an α-chain of a haemopoietin receptor capable of interaction with IL-13 with low affinity.

4. An isolated nucleic acid molecule according to claim 1 or 2 wherein the receptor is a derivative of an α-chain of a haemopoietin receptor capable of interaction with IL-13 with medium to high affinity.

5. An isolated nucleic acid molecule according to claim 1 or 2 encoding a receptor having an amino acid sequence substantially as set forth in SEQ ID NO:2 or SEQ ID NO:4 or having at least about 50% similarity to all or part thereof.

6. An isolated nucleic acid molecule according to claim 1 or 2 comprising a sequence of nucleotides substantially as set forth in SEQ ID NO:1 or SEQ ID NO:3 or having at least about 50% similarity to all or part thereof.

7. An isolated nucleic acid molecule comprising a sequence of nucleotides which encodes or is complementary to a sequence which encodes an IL-13 receptor α-chain or a derivative thereof, said nucleic acid molecule having a nucleotide sequence substantially as set forth in SEQ ID NO:1 or SEQ ID NO:3 or a nucleic acid molecule which encodes a functionally similar IL-13 receptor α-chain or a derivative thereof and which is capable of hybridising to the nucleotide sequence substantially as set forth in SEQ ID NO:1 or SEQ ID NO:3 or a complementary form thereof under low stringency conditions.

8. An isolated nucleic acid molecule comprising a sequence of nucleotides which encodes or is complementary to a sequence which encodes the IL-13 receptor α-chain or a derivative thereof having an amino acid sequence substantially as set forth in SEQ ID NO:2 or SEQ ID NO:4 or comprises a nucleotide sequence coding for an amino acid sequence having at least about 50% similarity to the sequence set forth in SEQ ID NO:2 or SEQ ID NO:4 and is capable of hybridising to the sequence set forth in SEQ ID NO:1 or SEQ ID NO:3 under low stringency conditions.

9. An isolated nucleic acid molecule according to claim 1 or 2 or 7 or 8 which encodes a haemopoietin receptor capable of interaction with IL-13 or its derivatives, which interaction is capable of competitive inhibition by IL-4 or a derivative thereof in cells which express an IL-4 receptor α-chain.

10. A genetic construct comprising a nucleic acid molecule according to claim 1 or 6 or 7 operably linked to a promoter capable of directing expression of said nucleic acid molecule in a host cell.

11. A recombinant polypeptide comprising a sequence of amino acids substantially as set forth in SEQ ID NO:2 or SEQ ID NO:4 or having at least about 50% similarly to all or part thereof, said polypeptide capable of interaction with IL-13 or its derivatives.

12. A recombinant polypeptide according to claim 11 wherein the interaction with IL-13 is competitively inhibited by IL-4 in cells which express an IL-4 receptor α-chain.

13. A recombinant polypeptide according to claim 11 wherein the interaction with IL-13 is with low affinity.

14. A recombinant polypeptide according to claim 10 wherein the interaction with IL-13 is with medium to high affinity.

15. A recombinant polypeptide according claim 11 wherein said polypeptide has a molecular weight of from about 50,000 to about 70,000 daltons as determined by Western blot analysis when expressed in COS cells.

16. A recombinant polypeptide having at least two of the following characteristics:
(i) comprises an amino acid sequence substantially as set forth in SEQ ID NO:2 or SEQ ID NO:4 or having at least about 50% similarity to all or part thereof;
(ii) is encoded by a nucleotide sequence substantially as set forth in SEQ ID NO:1 or SEQ ID NO:3 or having at least about 50% similarity to all or part thereof;
(iii) interacts with IL-13 or its derivatives with at least low affinity; and
(iv) has a molecular weight of from about 50,000 to about 70,000 daltons as determined by Western blot analysis when expressed in COS cells.

17. A recombinant polypeptide having at least three of the following characteristics:
(i) comprises an amino acid sequence substantially as set forth in SEQ ID NO:2 or SEQ ID NO:4 or having at least about 50% similarity to all or part thereof;
(ii) is encoded by a nucleotide sequence substantially as set forth in SEQ ID NO:1 or SEQ ID NO:3 or having at least about 50% similarity to all or part thereof;
(iii) interacts with IL-13 or its derivatives with at least low affinity;
(iv) has a molecular weight of from about 50,000 to about 70,000 daltons as determined by Western blot analysis when expressed in COS cells;
(v) comprises an amino acid sequence derived from IL-4 receptor α-chain; and
(vi) is capable of interaction with IL-13 which is competitively inhibited by IL-4 in cells which express an IL-4 receptor α-chain.

18. An antibody to the recombinant polypeptide according to claim 16 and 17.

19. An antibody according to claim 16 wherein said antibody is a monoclonal antibody.

20. A hybrid haemopoietin receptor capable of interaction with at least two cytokines wherein at least one of said cytokines is IL-13 or its derivatives and wherein said hybrid receptor comprises an amino acid sequence which includes all or part of the amino acid sequence set forth in SEQ ID NO:2 or SEQ ID NO:4.

21. A hybrid haemopoietin receptor capable of high affinity interaction with at least one cytokine wherein at least one of said cytokines is IL-13 or its derivatives and wherein said hybrid receptor comprises an amino acid sequence which includes all or part of the amino acid sequence set forth in SEQ ID NO:2 or SEQ ID NO:4.

22. A hybrid haemopoietic receptor according to claim 18 capable of interaction with IL-4.

23. A hybrid haemopoietin receptor according to claim 21 capable of interaction with a cytokine selected from IL-2, IL-5, IL-7, IL-9 and IL-15.

24. A pharmaceutical composition comprising a recombinant polypeptide according to claim 16 or 17 and one or more pharmaceutically acceptable carriers and/or diluents.

25. A genetic pharmaceutical composition comprising a nucleic acid molecule according to claim 1 or 2 or 7 or 8 and one or more genetically acceptable carriers and/or diluents.

26. A method of treatment in an animal comprising administering to said animal a treatment effective amount of a recombinant polypeptide according to claim 16 or 17.

27. A method of treating asthma, allergy or a condition exacerbated by IgE production in an animal comprising administering to said animal a treatment of an effective amount of a recombinant polypeptide according to claim 16 or 17.

28. A method of producing a recombinant polypeptide having at least two of the following characteristics:
(i) comprises an amino acid sequence substantially as set forth in SEQ ID NO:2 or SEQ ID NO:4 or having at least about 50% similarity thereto;
(ii) is encoded by a nucleotide sequence substantially as set forth in SEQ ID NO:1 or SEQ ID NO:3 or having at least about 50% similarity thereto;
(iii) interacts with IL-13 or its derivatives with at least low affinity; and
(iv) has a molecular weight of from about 50,000 to about 70,000 daltons as determined by Western blot analysis when expressed in COS cells,
said method comprising culturing cells comprising the genetic construct according to claim 10 for a time and under conditions sufficient to express the nucleic acid molecule in said genetic construct to produce a recombinant polypeptide and isolating said recombinant polypeptide.

29. A method of producing a recombinant polypeptide having at least three of the following characteristics:
(i) comprises an amino acid sequence substantially as set forth in SEQ ID NO:2 or SEQ ID NO:4 or having at least about 50% similarity to all or part thereof;
(ii) is encoded by a nucleotide sequence substantially as set forth in SEQ ID NO:1 or SEQ ID NO:3 or having at least about 50% similarity to all or part thereof;
(iii) interacts with IL-13 or its derivatives with at least low affinity;
(iv) has a molecular weight of from about 50,000 to about 70,000 daltons as determined by Western blot analysis when expressed in COS cells;
(v) comprises an amino acid sequence derived from IL-4 receptor α-chain; and
(vi) is capable of interaction with IL-13 which is competitively inhibited by IL-4 in cells which express an IL-4 receptor α-chain.
said method comprising culturing cells comprising the genetic construct according to claim 10 for a time and under conditions sufficient to express the nucleic acid molecule in said genetic construct to produce a recombinant polypeptide and isolating said recombinant polypeptide.

30. Animal cells which express the recombinant polypeptide produced by the method according to claim 28 and 29.

31. A chimeric protein comprising a first portion capable of interaction with IL-13 or its derivatives and a second portion derived from a haempoietin receptor, a receptor tyrosine kinase, a TNF/NGF receptor or a G protein coupled receptor.

32. A chimeric protein according to claim 31 wherein the second portion comprises all or a functional portion of IL-13 binding protein, IL-4 receptor α-chain, IL-2 receptor γ-chain or a receptor for a cytokine implicated in asthma or allergy.

33. A method for monitoring the level of IL-4 in a biological sample said method comprising incubating said biological sample with cells which express NR4 and IL-4 receptor α-chain together with an effective amount of IL-13 to competitively inhibit IL-4 binding to its receptor and determining the extent of competitive inhibition.

34. A method for monitoring the level of IL-13 in a biological sample said method comprising incubating said biological sample with cells which express NR4 and IL-4 receptor α-chain together with an effective amount of IL-4 to competitively inhibit IL-13 binding to its receptor and determining the extent of competitive inhibition.

35. A method according to claim 33 or 34 wherein the cytokines are labelled with a reporter molecule.
